(19) United States
(12) Patent Application Publication
(10) Pub. No.: US 2015/0056158 A1
(43) Pub. Date: Feb. 26, 2015

(54) BRANCHED POLYMERS

(71) Applicant: Commonwealth Scientific and Industrial Research Organisation, Campbell (AU)

(72) Inventors: Pathiraja Arachchillage Gunatillake, Mulgrave (AU); Tracey Michelle Hinton, Belmont (AU); San Thang, Camberwell (AU); Mark Leslie Tizard, Highton (AU)

(21) Appl. No.: 14/376,360

(22) PCT Filed: Feb. 1, 2013
(86) PCT No.: PCT/AU2013/000091

§ 371 Date: Aug. 1, 2014

(30) Foreign Application Priority Data
Feb. 3, 2012 (AU) 2012900396

(51) Int. Cl. C08F 293/00; C12N 15/113; A61K 47/48

(52) U.S. Cl. CPC C08F 293/005; A61K 47/48176; C12N 15/1131; C12N 2320/32; C12N 2310/14; C12N 2310/351
USPC 424/78.29; 525/299; 525/54.2; 435/455; 435/471; 435/468

(57) ABSTRACT

The present invention relates to branched polymer comprising a support moiety and at least three block co-polymer chains covalently coupled to and extending from the moiety, wherein: (i) each of the at least three block co-polymer chains comprise (a) a cationic polymer block that is covalently coupled to a hydrophilic polymer block, or (b) a cationic polymer block that is covalently coupled to a hydrophobic polymer block, said hydrophobic polymer block being covalently coupled to a hydrophilic polymer block; and (ii) at least one of said covalent couplings associated with each of said block co-polymer chains is biodegradable.

### FIELD OF THE INVENTION

The present invention relates in general to branched polymers. More particularly, the invention relates to polymers having at least three block copolymer arms. The polymers are particularly suited for use in forming complexes with nucleic acid molecules, and it will therefore be convenient to describe the invention with an emphasis toward this application. However, it is to be understood that the polymers may be used in various other applications. The invention therefore also relates to a complex of a nucleic acid molecule and the polymer, to the use of such complexes in a method of delivering a nucleic acid molecule to cells, and to a method of silencing gene expression. The invention further relates to the use of the polymer in a method of protecting a nucleic acid molecule from enzymatic degradation, and to reagents for preparing the polymers.

### BACKGROUND OF THE INVENTION

Branched polymers are a type of polymer known in the art to comprise a support moiety such as an atom or molecule to which is attached at least three polymer chains. The at least three polymer chains may be referred to as the “arms” of the branched polymer. Specific types of branched polymer include star polymers, comb polymers, brush polymers and dendrimers. Such polymer structures afford different physical and chemical properties compared with linear polymers and are therefore of considerable theoretical and practical interest.

The properties of branched polymers are by in large influenced by their molecular architecture and composition. Through manipulation of their molecular structure, branched polymers have been found to exhibit a variety of unique properties and have been employed in a diverse array of applications functioning, for example, as elastomers, surfactants and lubricants.

Accordingly, there remains an opportunity for developing new branched polymer structures that exhibit properties suitable for further extending their utility.

### SUMMARY OF THE INVENTION

The present invention therefore provides branched polymer comprising a support moiety and at least three block co-polymer chains covalently coupled to and extending from the moiety, wherein:
(i) each of the at least three block co-polymer chains comprise (a) a cationic polymer block that is covalently coupled to a hydrophilic polymer block, or (b) a cationic polymer block that is covalently coupled to a hydrophobic polymer block, said hydrophobic polymer block being covalently coupled to a hydrophilic polymer block; and
(ii) at least one of said covalent couplings associated with each of said block co-polymer chains is biodegradable.

It has now been found that branched polymers according to the present invention present a unique combination of at least cationic, hydrophilic and biodegradable features that enable them to undergo a substantive structural transformation when subjected to a biodegrading environment. This structural transformation involves cleavage (at a biodegradable covalent coupling present in each of the at least three block co-polymer chains) of (i) an entire block co-polymer chain, (ii) part of a block co-polymer chain, or (iii) a combination thereof.

Where only part of a block co-polymer chain is cleaved, the branched polymer may loose a hydrophilic polymer block, a cationic polymer block, or a hydrophobic polymer block (when present), or when all three of such polymer blocks are present in a chain, a combination of two of these polymer blocks.

Loss from the branched polymer of a chain or part thereof advantageously promotes a change in the polymer's properties such as its hydrophilic, hydrophobic, and/or cationic character. The molecular weight of the branched polymer will of course also be reduced.

In one embodiment, each of the covalent couplings that couple the at least three block co-polymer chains to the support moiety are biodegradable.

Through appropriate selection of biodegradable covalent couplings, the branched polymer can be designed to undergo specific structural transformations in a particular biodegrading environment. For example, the branched polymer may be designed to form a complex with a nucleic acid molecule, where upon the complex undergoing transfection all or part of the chains of the branched polymer are cleaved at the biodegradable covalent couplings. This structural transformation of the branched polymer within the cell may provide for enhanced availability of the nucleic acid molecule and also facilitate metabolism and clearance of the branched polymer (or its residues).

Due to the block nature of the chains, the branched polymers according to the invention can advantageously be tailor-designed to suit a variety of applications. Through selection of at least appropriate cationic and hydrophilic blocks that form the chains, the branched polymer can, for example, be designed to effectively and efficiently form a complex with a nucleic acid molecule.

The present invention therefore also provides a complex comprising a branched polymer and a nucleic acid molecule, the branched polymer comprising a support moiety and at least three block co-polymer chains covalently coupled to and extending from the moiety, wherein:
(i) each of the at least three block co-polymer chains comprise (a) a cationic polymer block that is covalently coupled to a hydrophilic polymer block, or (b) a cationic polymer block that is covalently coupled to a hydrophobic polymer block, said hydrophobic polymer block being covalently coupled to a hydrophilic polymer block; and
(ii) at least one of said covalent couplings associated with each of said block co-polymer chains is biodegradable.

In this context, it will be appreciated that the complex per se is from between the branched polymer and the nucleic acid molecule.

The branched polymer can form stable complexes with a variety of nucleic acid molecules, with the resulting complex affording improved transfection for a nucleic acid molecule to a variety of cell types. The branched polymers, when in the form of the complex, have also been found to afford good protection to nucleic acid molecules from enzymatic degradation.

Due to their block character, each arm of the branched polymer can advantageously be tailor-designed to provide for efficient complexation with a given nucleic acid molecule and/or for efficient transfection of the nucleic acid molecule with a given cell type. The branched polymer can also advantageously be tailor-designed to incorporate a targeting ligand that directs the complex to a chosen targeted cell type.

In one embodiment, each of the at least three block co-polymer chains comprise a cationic polymer block that is covalently coupled to a hydrophilic polymer block. In that case, the block copolymer chains may be conveniently referred to as having an A-B di-block structure, where A represents the hydrophilic polymer block, and B represents the cationic polymer block. A further polymer block, such as a hydrophobic polymer block, may be covalently coupled to either the cationic polymer block or the hydrophilic polymer block. In that case, the block copolymer chains may be conveniently referred to as having an A-B-C or C-A-B tri-block structure, where A represents the hydrophilic polymer block, B represents the cationic polymer block, and C represents a further polymer block such as a hydrophobic polymer block.

In another embodiment, each of the at least three block co-polymer chains comprise two hydrophilic polymer blocks and a cationic polymer block, where the cationic polymer block is (i) located in between, and (ii) covalently coupled to, each of the two hydrophilic polymer blocks. In that case, the block copolymer chains may be conveniently referred to as having an A-B-A tri-block structure, where each A may be the same or different and represents a hydrophilic polymer block, and B represents the cationic polymer block.

In a further embodiment, each of the at least three block co-polymer chains comprise a hydrophilic polymer block and two cationic polymer blocks, where the hydrophilic polymer block is (i) located in between, and (ii) covalently coupled to, each of the two cationic polymer blocks. In that case, the block copolymer chains may be conveniently referred to as having a B-A-B tri-block structure, where each B may be the same or different and represents a cationic polymer block, and A represents the hydrophilic polymer block.

In another embodiment, each of the at least three block co-polymer chains comprise a cationic polymer block that is covalently coupled to a hydrophobic polymer block, the hydrophobic polymer block itself being covalently coupled to a hydrophilic polymer block. In that case, the block copolymer arms may be conveniently referred to as having a B-C-A tri-block structure, where B represents the cationic polymer block, C represents the hydrophobic polymer block, and A represents the hydrophilic polymer block.

The present invention also provides a method of delivering a nucleic acid molecule to a cell, the method comprising:

(a) providing a complex comprising a branched polymer and a nucleic acid molecule, the branched polymer comprising a support moiety and at least three block co-polymer chains covalently coupled to and extending from the moiety, wherein:
(i) each of the at least three block co-polymer chains comprise (a) a cationic polymer block that is covalently coupled to a hydrophilic polymer block, or (b) a cationic polymer block that is covalently coupled to a hydrophobic polymer block, said hydrophobic polymer block being covalently coupled to a hydrophilic polymer block; and
(ii) at least one of said covalent couplings associated with each of said block co-polymer chains is biodegradable; and
   (b) delivering the complex to the cell.

In one embodiment, the nucleic acid molecule is delivered to a cell for the purpose of silencing gene expression.

The present invention therefore also provides a method of silencing gene expression, the method comprising transfecting a cell with a complex comprising a branched polymer and a nucleic acid molecule, the branched polymer comprising a support moiety and at least three block co-polymer chains covalently coupled to and extending from the moiety, wherein:
(i) each of the at least three block co-polymer chains comprise (a) a cationic polymer block that is covalently coupled to a hydrophilic polymer block, or (b) a cationic polymer block that is covalently coupled to a hydrophobic polymer block, said hydrophobic polymer block being covalently coupled to a hydrophilic polymer block; and
(ii) at least one of said covalent couplings associated with each of said block co-polymer chains is biodegradable.

In one embodiment of this and other aspects of the invention, the nucleic acid molecule is selected from DNA and RNA.

In a further embodiment, the DNA and RNA are selected from gDNA, cDNA, double or single stranded DNA oligonucleotides, sense RNAs, antisense RNAs, mRNAs, tRNAs, rRNAs, small/short interfering RNAs (siRNAs), double-stranded RNAs (dsRNA), short hairpin RNAs (shRNAs), piwi-interacting RNAs (PiRNA), micro RNA/small temporal RNA (miRNA/stRNA), small nucleolar RNAs (SnoRNAs), small nuclear (SnRNAs) ribozymes, aptamers. DNAzymes, ribonuclease-type complexes, hairpin double stranded RNA (hairpin dsRNA), miRNAs which mediate spatial development (sdRNAs), stress response RNA (srRNAs), cell cycle RNA (ccRNAs) and double or single stranded RNA oligonucleotides.

Branched polymers in accordance with the invention have also been found to protect nucleic acid molecules against enzymatic degradation.

The present invention therefore also provides a method of protecting a nucleic acid molecule form enzymatic degradation, the method comprising complexing the nucleic acid molecule with a branched polymer comprising a support moiety and at least three block co-polymer chains covalently coupled to and extending from the moiety, wherein:
(i) each of the at least three block co-polymer chains comprise (a) a cationic polymer block that is covalently coupled to a hydrophilic polymer block, or (b) a cationic polymer block that is covalently coupled to a hydrophobic polymer block, said hydrophobic polymer block being covalently coupled to a hydrophilic polymer block; and
(ii), at least one of said covalent couplings associated with each of said block co-polymer chains is biodegradable.

There is also provided use of a complex for delivering a nucleic acid molecule to a cell, the complex comprising a branched polymer and the nucleic acid molecule, the branched polymer comprising a support moiety and at least three block co-polymer chains covalently coupled to and extending from the moiety, wherein:
(i) each of the at least three block co-polymer chains comprise (a) a cationic polymer block that is covalently coupled to a hydrophilic polymer block, or (b) a cationic polymer block that is covalently coupled to a hydrophobic polymer block, said hydrophobic polymer block being covalently coupled to a hydrophilic polymer block; and
(ii) at least one of said covalent couplings associated with each of said block co polymer chains is biodegradable.

The present invention further provides use of a complex for silencing gene expression, the complex comprising a branched polymer and a nucleic acid molecule, the branched polymer comprising a support moiety and at least three block co-polymer chains covalently coupled to and extending from the moiety, wherein:
(i) each of the at least three block co-polymer chains comprise (a) a cationic polymer block that is covalently coupled to a hydrophilic polymer block, or (b) a cationic polymer block that is covalently coupled to a hydrophobic polymer block, said hydrophobic polymer block being covalently coupled to a hydrophilic polymer block; and
(ii) at least one of said covalent couplings associated with each of said block co-polymer chains is biodegradable.

The present invention further provides use of a branched polymer in protecting a nucleic acid molecule from enzymatic degradation, the branched polymer comprising a support moiety and at least three block co-polymer chains covalently coupled to and extending from the moiety, wherein:
(i) each of the at least three block co-polymer chains comprise (a) a cationic polymer block that is covalently coupled to a hydrophilic polymer block, or (b) a cationic polymer block that is covalently coupled to a hydrophobic polymer block, said hydrophobic polymer block being covalently coupled to a hydrophilic polymer block; and
(ii) at least one of said covalent couplings associated with each of said block co-polymer chains is biodegradable.

Further aspects and embodiments of the invention appear below in the detailed description of the invention.

### BRIEF DESCRIPTION OF THE DRAWINGS

The invention will herein be described with reference to the following non-limiting drawings in which:

FIG. 1 illustrates a variety of branched polymer structures that may be formed in accordance with the invention, where [symbol] represents the support moiety, ______ represents a general covalent bond, [symbol] represents a biodegradable covalent coupling or linking moiety, [symbol] represents a cationic polymer block, [symbol] and represents a hydrophilic polymer block;

FIG. 2 illustrates the viability of CHO-GFP and HEK293T cells exposed to multi-arm star copolymer serial dilutions prepared in Examples 1 and 2 (without siRNA);

FIG. 3 illustrates the viability of CHO-GFP, HEK293T and Huh7-GFP cells exposed to multi-arms star copolymers prepared in Examples 1 and 2 (with siRNA);

FIG. 4 illustrates the association of multi-arms star copolymers with siRNA as a function of polymer: siRNA ratio (w/w) for the series of polymers prepared in Example 1. Also shown is the corresponding N/P ratio;

FIG. 5 illustrates gene silencing in CHO-GFP, HEK-GFP and Huh7-GFP cells for different siRNA:RAFT polymer (prepared in Example 5) combinations presented as a percentage of L2000 di22 samples or polymer/di22 complexes mean EGFP fluorescence;

FIG. 6 illustrates the uptake of fluorescently labelled si22/ToTo-3 polymer complexes in CHO-GFP cells. CHO-GFP cells were transfected with 50 pmole of siRNA labelled with ToTo-3 with Lipofectamine 2000 as a positive control or had 4:1 molar ratio of polymer:siRNA labelled with ToTo-3 added for 6 or 24 h. Naked si22 labelled with ToTo-3 was also added. Cells were then assayed by flow cytometry and analysed. Values are shown as uptake index compared to naked si22 labelled with ToTo-3, the graph is representative of three separate experiments in triplicate±standard deviation;

FIG. 7 illustrates the stability of siRNA/polymer complexes in foetal bovine serum (FBS); (a) stability of naked siRNA, (b) ABA-B4S-16/24, (C) BAB-B4S-11/10 (d) ability of the treated complexes to silence in CHO-GFP cells;

FIG. 8 illustrates interference response to ABA-B4S-16/24 in chicken embryo (A) Intererence alpha (IFNα) and (B) interference beta (IFNβ). Histology sections of liver (C, D and E) after 24 h; and

FIG. 9 illustrates uptake of ABA-B4S-16/24 di22-FAM complexes in Chicken Embryos at 24 h (A & B) and influenza virus inhibition in chicken embryos (C).

Some Figures contain colour representations or entities. Coloured versions of the Figures are available upon request.

### DETAILED DESCRIPTION OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

As used herein, the singular forms “a”, “and” and “the” are intended to include plural aspects unless the context clearly dictates otherwise. Thus, for example, reference to “a cell” includes a single cell as well as two or more cells; reference to “an agent” includes one agent, as well as two or more agents; and so forth.

As used herein the expression “branched polymer” is intended to mean polymer that comprises a support moiety to which is attached at least three polymer chains. The polymer may comprise more than one of such support moieties. For convenience, the at least three polymer chains may be referred to as “arms” of the branched polymer. The branched polymer may have more than three of such arms. For example, the branched polymer may have 4, 5, 6, 7, 8, 9, 10 or more polymer chains attached to the support moiety.

Specific types of branched polymer include, but are not limited to, star polymers, comb polymers, brush polymers and dendrimers.

The at least three polymer chains or arms attached to the support moiety may be branched or linear polymer chains.

In one embodiment, the at least three polymer chains attached to the support moiety are linear polymer chains.

In a further embodiment, the branched polymer in accordance with the invention is a star polymer.

By “star polymer” is meant a macromolecule comprising a single branch moiety from which emanate at least three covalently coupled linear polymer chains or arms. In that case, the branch moiety represents the support moiety, and the support moiety may be in the form of a suitable atom or a molecule as herein described.

By “support moiety” is meant a moiety, such as an atom or molecule, to which is covalently attached the arms of the branched polymer. Accordingly, the support moiety functions to support the covalently attached arms.

To assist with describing what is intended by the expressions “branched polymer” and “support moiety”, reference may be made to general formula (A) below:

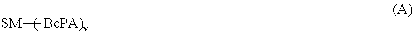

where SM represents the support moiety, BcPA represents the block co-polymer arm, and v is an integer greater than or equal to 3.

With reference to general formula (A), the support moiety (SM) has at least three block co-polymer arms (BcPA) covalently coupled thereto, and as such, SM may simplistically be viewed as a structural feature from which branching can occur. The branched polymer in accordance with the invention may have more such structural features from which branching can occur.

Where the support moiety is an atom, it will generally be C, Si or N. In the case where the atom is C or Si, there may be a fourth block co-polymer chain covalently coupled to the respective atom.

Where the support moiety is a molecule, there is no particular limitation concerning the nature of the moiety provided it can have the at least three block co-polymer arms covalently coupled to it. In other words, the molecule must be at least tri-valent (i.e. have at least three points at which covalent attachment occurs). For example, the molecule can be selected from at least tri-valent forms of optionally substituted: alkyl, alkenyl, alkynyl, aryl, carbocyclyl, heterocyclyl, heteroaryl, alkyloxy, alkenyloxy, alkynyloxy, aryloxy, carbocyclyloxy, heterocyclyloxy, heteroaryloxy, alkylthio, alkenylthio, alkynylthio, arylthio, carbocyclylthio, heterocyclylthio, heteroarylthio, alkylalkenyl, alkylalkynyl, alkylaryl, alkylaryl, alkylcarbocyclyl, alkylheterocyclyl, alkylheteroaryl, alkyloxyalkyl, alkenyloxyalkyl, alkynyloxyalkyl, aryloxyalkyl, alkylacyloxy, alkyloxyacylalkyl, alkylcarbocyclyloxy, alkylheterocyclyloxy, alkylheteroaryloxy, alkylthioalkyl, alkenylthioalkyl, alkynylthioalkyl, arylthioalkyl, alkylacylthio, alkylcarbocyclylthio, alkylheterocyclylthio, alkylheteroarylthio, alkylalkenylalkyl, alkylalkynylalkyl, alkylarylalkyl, alkylacylalkyl, arylalkylaryl, arylalkenylaryl, arylalkynylaryl, arylacylaryl, arylacyl, arylcarbocyclyl, arylheterocyclyl, arylheteroaryl, alkenyloxyaryl, alkynyloxyaryl, aryloxyaryl, arylacyloxy, arylcarbocyclyloxy, arylheterocyclyloxy, arylheteroaryloxy, alkylthioaryl, alkenylthioaryl, alkynylthioaryl, arylthioaryl, arylacylthio, arylcarbocyclylthio, arylheterocyclylthio, arylheteroarylthio, coordination complex, and a polymer chain, wherein where present the or each —CH₂— group in any alkyl chain may be replaced by a divalent group independently selected from —O—, —OP(O)₂—, —OP(O)₂O—, —S—, —S(O)—, —S(O)₂O—, —OS(O)₂O—, —N═N—, —OSi(OR^{a})₂O—, —Si(OR^{a})₂O—, —OB(OR^{a})O—, —B(OR^{a})O—, —NR^{a}—, —C(O)—, —C(O)O—, —OC(O)O—, —OC(O)NR^{a}— and —C(O)NR^{a}—, where the or each R^{a} may be independently selected from hydrogen, alkyl, alkenyl, alkynyl, aryl, carbocyclyl, heteroaryl, heterocyclyl, arylalkyl, and acyl. The or each R^{a} may also be independently selected from hydrogen, C₁₋₁₈alkyl, C₁₋₁₈alkenyl, C₁₋₁₈alkynyl, C₆₋₁₈aryl, C₃₋₁₈carbocyclyl, C₃₋₁₈heteroaryl, C₃₋₁₈heterocyclyl, and C₇₋₁₈arylalkyl.

Where the branched polymer comprises only one support moiety and the at least three block co-polymer arms are linear, the branched polymer may be conveniently referred to as a star polymer.

When defining the support moiety it can be convenient to refer to a compound from which the moiety is derived. For example, the support moiety may be derived from a compound having three or more functional groups that provide reactive sites through which the block co-polymer arms are to be covalently coupled. In that case, the support moiety may be derived from a compound having three or more functional groups selected from, for example, halogen, alcohol, thiol, carboxylic acid, amine, epoxide, and acid chloride.

Examples of compounds having three or more alcohol functional groups from which the support moiety may be derived include, but are not limited to, glycerol, pentaerythritol, dipentaerythritol, tripentaerythritol, 1,2,3-trihydroxyhexane, trimethylolpropane, myoinisitol, glucose and its isomers (e.g. d-galactose, d-manose, d-fructose), maltose, sucrose, and manitol.

The at least three block co-polymer chains are each covalently coupled to the support moiety. Each block co-polymer chain may be covalently coupled directly or indirectly to the support moiety. By being “directly” coupled is meant that there is only a covalent bond between the block co-polymer chain and the support moiety. By being “indirectly” coupled is meant that there is located between the block co-polymer chain and the support moiety one or more covalently bonded atoms or molecules. Where the block co-polymer chains are indirectly coupled to the support moiety, it may be convenient to refer to the block co-polymer chains as being covalent coupled to the support moiety through a linking moiety.

In one embodiment, each of the at least three block co-polymer chains are each covalently coupled to the support moiety through a linking moiety.

There is no particular limitation concerning the nature of such a linking moiety provided it can function to couple the at least three block co-polymer chains to the support moiety.

Examples of suitable linking moieties include a divalent form of optionally substituted: oxy (—O—), disulfide (—S—S—), alkyl; alkenyl, alkynyl, aryl, acyl (including —C(O)—), carbocyclyl, heterocyclyl, heteroaryl, alkyloxy, alkenyloxy, alkynyloxy, aryloxy, acyloxy, carbocyclyloxy, heterocyclyloxy, heteroaryloxy, alkylthio, alkenylthio, alkynylthio, arylthio, acylthio, carbocyclylthio, heterocyclylthio, heteroarylthio, alkylalkenyl, alkylalkynyl, alkylaryl, alkylacyl, alkylcarbocyclyl, alkylheterocyclyl, alkylheteroaryl, alkyloxyalkyl, alkenyloxyalkyl, alkynyloxyalkyl, aryloxyalkyl, alkylacyloxy, alkyloxyacylalkyl, alkylcarbocyclyloxy, alkylheterocyclyloxy, alkylheteroaryloxy, alkylthioalkyl, alkenylthioalkyl, alkynylthioalkyl, arylthioalkyl, alkylacylthio, alkylcarbocyclylthio, alkylheterocyclylthio, alkylheteroarylthio, alkylalkenylalkyl, alkylalkynylalkyl, alkylarylalkyl, alkylacylalkyl, arylalkylaryl, arylalkenylaryl, arylalkynylaryl, arylacylaryl, arylacyl, arylcarbocyclyl, arylheterocyclyl, arylheteroaryl, alkenyloxyaryl, alkynyloxyaryl, aryloxyaryl, arylacyloxy, arylcarbocyclyloxy, arylheterocyclyloxy, arylheteroaryloxy, alkylthioaryl, alkenylthioaryl, alkynylthioaryl, arylthioaryl, arylacylthio, arylcarbocyclylthio, arylheterocyclylthio, and arylheteroarylthio, wherein where present the or each —CH₂— group in any alkyl chain may be replaced by a divalent group independently selected from —O—, —OP(O)₂—, —OP(O)₂O—, —S—, —S(O)—, —S(O)₂O—, —OS(O)₂O—, —N═N—, —OSi(OR^{a})₂O—, —Si(OR^{a})₂O—, —OB(OR^{a})O—, —B(OR^{a})O—, —NR^{a}—, —C(O)—, —C(O)O—, —OC(O)O—, —OC(O)NR^{a}— and —C(O)NR^{a}—, where the or each R^{a} may be independently selected from hydrogen, alkyl, alkenyl, alkynyl, aryl, carbocyclyl, heteroaryl, heterocyclyl, arylalkyl, and acyl. The or each R^{a} may also be independently selected from hydrogen, C₁₋₁₈alkyl, C₁₋₁₈alkenyl, C₁₋₁₈alkynyl, C₆₋₁₈aryl, C₃₋₁₈carbocyclyl, C₃₋₁₈heteroaryl, C₃₋₁₈heterocyclyl, and C₇₋₁₈arylalkyl.

Reference herein to groups containing two or more subgroups (e.g. [group A][group B]), are not intended to be limited to the order in which the subgroups are presented. Thus, two subgroups defined as [group A][group B] (e.g. alkylaryl) is intended to also be a reference to two subgroups defined as [group B][group A] (e.g. arylalkyl).

An important feature of the present invention is that at least one of the covalent couplings associated with each of the block co-polymer chains is biodegradable.

In one embodiment, each of the at least three block co-polymer chains are each covalently coupled to the support moiety through a boidegradable linking moiety

By a covalent couple(s), covalent coupling(s), or linking moiety being “biodegradable” is meant that it has a molecular structure that is susceptible to break down (i.e. undergoing bond cleavage) via a chemical reaction upon being exposed to a biological environment (e.g. within a subject or in contact with biological material such as blood, tissue etc) such that the relevant covalent coupling (e.g. between the support moiety and the block copolymer chain) is severed. Such chemical decomposition may be via hydrolysis, oxidation or reduction. Accordingly, biodegradable covalent couple(s), covalent coupling(s), or linking moieties will generally be susceptible to undergoing hydrolytic, oxidative or reductive cleavage. The rate of biodegradation may vary depending on a number of extrinsic or intrinsic factors (e.g. light, heat, radiation, pH, enzymatic or nonenzymatic mediation, etc.).

Where a biodegradable linking moiety is used to covalently couple a block co-polymer chain to the support moiety, it will be appreciated that the so coupled block co-polymer chain will itself be cleaved from the branched polymer structure at the time when the biodegradable linking moiety undergoes biodegradation.

Those skilled in the art will appreciate the type of functional groups that can form, or form part of, a linking moiety to render it susceptible to undergoing biodegradation. Such functional groups may include, for example, ester, anhydride, carbonate, peroxide, peroxyester, phosphate, thioester, urea, thiourethane, ether, disulfide, carbamate (urethane) and boronate ester.

In one embodiment the biodegradable linking moiety comprises one or more functional groups selected from ester, anhydride, carbonate, peroxide, peroxyester, phosphate, thioester, urea, thiourethane, ether, disulfide, carbamate (urethane) and boronate ester.

It will be appreciated that such functional groups will be located within the linking moiety such that upon undergoing biodegradation the relevant covalent coupling is severed. Such functional groups therefore directly form part of the string of atoms that provide the covalent coupling. In other words, at least one atom of such functional groups is present in the direct string of atoms that covalently couple the relevant sections of the polymer (e.g. the support moiety to the block co-polymer chains).

Accordingly, the linking moiety may be biodegradable through one or more functional groups selected from ester, anhydride, carbonate, peroxide, peroxyester, phosphate, thioester, urea, thiourethane, ether, disulfide, carbamate (urethane) and boronate ester.

Biodegradation of the biodegradable linking moiety may be facilitated in the presence of an acid, a base, an enzyme and/or another endogenous biological compound that can catalyze or at least assist in the bond cleavage process. For example, an ester may be hydrolytically cleaved to produce a carboxylic acid group and an alcohol group, an amide may be hydrolytically cleaved to produce a carboxylic acid group and an amine group, and a disulfide may be reductively cleaved to produce thiol groups.

Biodegradation may occur in a biological fluid such as blood, plasma, serum, urine, saliva, milk, seminal fluid, vaginal fluid, synovial fluid, lymph fluid, amniotic fluid, sweat, and tears; as well as an aqueous solution produced by a plant, including, for example, exudates and guttation fluid, xylem, phloem, resin, and nectar.

Biodegradation may also occur in a cell or in cellular components such as endosomes and cytoplasm.

Biodegradable linking moieties may be selected such that they can undergo biodegradation upon being exposed to a particular biological environment. For example, a redox potential gradient exists between extracellular and intracellular environments in normal and pathophysiological states. Disulfide bonds present in a biodegradable linking moiety may be readily reduced in the reducing intracellular environment, while remaining intact in the oxidizing extracellular space. The intracellular reduction of the disulfide bond is typically executed by small redox molecules such as glutathione (GSH) and thioredoxin, either alone or with the help of enzymatic machinery.

A given biodegradable linking moiety may comprise two or more functional groups that render it susceptible to undergoing biodegradation. However, depending on the nature of these functional groups and the biodegradation environment, it may be that only one of the functional groups actually promotes the desired bond cleavage. For example, a biodegradable linking moiety may comprise ester and disulfide functional groups. In a reductive environment, it may be that only the disulfide functional group will undergo biodegradation. In a hydrolytic environment it may be that only the ester functional group will undergo biodegradation. In a reductive and hydrolytic environment it may be that both the disulfide and ester functional groups will undergo biodegradation.

The at least three block co-polymer chains that are covalently coupled to the support moiety comprise (a) a cationic polymer block that is covalently coupled to a hydrophilic polymer block, or (b) a cationic polymer block that is covalently coupled to a hydrophobic polymer block, where the hydrophobic polymer block is itself coupled to a hydrophilic polymer block.

In one embodiment, each of the at least three block co-polymer chains comprise a cationic polymer block that is covalently coupled to a hydrophilic polymer block. In that case, the block copolymer chains may be conveniently referred to as having an A-B di-block structure, where A represents the hydrophilic polymer block, and B represents the cationic polymer block. A further polymer block, such as a hydrophobic polymer block, may be covalently coupled to either the cationic polymer block or the hydrophilic polymer block. In that case, the block copolymer chains may be conveniently referred to as having an A-BC or C-A-B tri-block structure, where A represents the hydrophilic polymer block. B represents the cationic polymer block, and C represents a further polymer block such as a hydrophobic polymer block.

Where each of the at least three block co-polymer chains comprise a cationic polymer block that is covalently coupled to a hydrophilic polymer block, the cationic polymer block of each arm may be covalently coupled to the support moiety, or the hydrophilic polymer block of each arm may be covalently coupled to the support moiety. Where a further polymer block, such as a hydrophobic polymer block, is covalently coupled to either the cationic polymer block or the hydrophilic polymer block, the cationic polymer block, the hydrophilic polymer block, or the hydrophobic polymer block of each arm may be covalently coupled to the support moiety.

The hydrophilic polymer block, the cationic polymer block, or if present a further polymer block, such as a hydrophobic polymer block, may in the desired order be directly or indirectly covalently coupled to each other.

In a similar fashion to that outlined above in describing the nature of the covalent coupling between each block copolymer chain and the support moiety, by being “directly” coupled in the context of at least two blocks within the block copolymer chain is meant that there is only a covalent bond between the respective polymer blocks. Also, by being “indirectly” coupled in the context of at least two blocks within the block copolymer chain is meant that there is located between the respective blocks one or more covalently bonded atoms or molecules. Where two or more blocks within the block copolymer chain are indirectly coupled, it may be convenient to refer to the respective blocks as being covalent coupled to each other through a linking moiety.

For example, each of the at least three block co-polymer chains may comprise a cationic polymer block that is covalently coupled through a linking moiety to a hydrophilic polymer block. Also, a further polymer block, such as a hydrophobic polymer block, may be covalently coupled through a linking moiety to either the cationic polymer block or the hydrophilic polymer block.

Where a given polymer block within a block copolymer chain is covalently coupled through a linking moiety to another polymer block, those skilled in the art will appreciate that despite the presence of such the linking moiety in between each polymer block, the overall structure will nevertheless be described as a block copolymer chain. For example, each of the at least three block co-polymer chains may comprise a cationic polymer block that is covalently coupled through a linking moiety to a hydrophilic polymer block. In that case, the block copolymer chains may be illustrated as having the structure A-LM-B, which in turn can be conveniently referred to as having an A-B di-block structure, where A represents the hydrophilic polymer block, LM represent the linking moiety and B represents the cationic polymer block.

In one embodiment, each of the at least three block co-polymer chains comprise a cationic polymer block that is covalently coupled through a linking moiety to a hydrophilic polymer block.

In a further embodiment, each of the at least three block co-polymer chains comprise a further polymer block, such as a hydrophobic polymer block, that is covalently coupled through a linking moiety to either the cationic polymer block or the hydrophilic polymer block.

Linking moieties described herein are suitable for covalently coupling the polymer blocks within each block copolymer chain.

In one embodiment, each of the at least three block co-polymer chains comprise a cationic polymer block that is covalently coupled through biodegradable linking moiety to a hydrophilic polymer block.

In a further embodiment, each of the at least three block co-polymer chains comprise a further polymer block, such as a hydrophobic polymer block, that is covalently coupled through a biodegradable linking moiety to either the cationic polymer block or the hydrophilic polymer block.

According to such embodiments, the branched polymer may be conveniently represented by formulae (A1)-(A6) below:

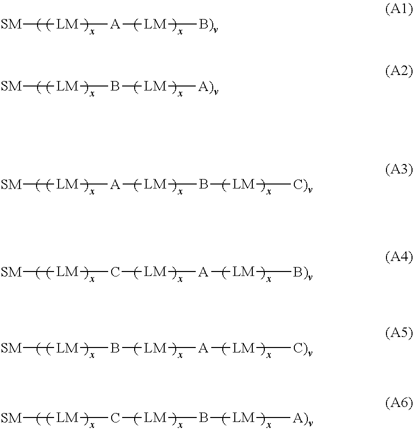

where SM represents the support moiety, LM represents a linking moiety, A represents a hydrophilic polymer block, B represents a cationic polymer block, C represents a further polymer block (such as a hydrophobic polymer block), each x is independently 0 or 1, and v is an integer greater than or equal to 3, such that (i) A, B, optionally together with LM and C, represent a block co-polymer arm of the branched polymer, and (ii) in each of the at least 3 block co-polymer arms at least one x=1 and the LM associated with that x=1 is a biodegradable linking moiety.

In structures A1-A6 above, it will be appreciated that where x=0 in a given block copolymer arm, the linking moiety (LM) is not present and the relevant parts of the branched polymer are directly covalently coupled to each other.

In structures A1-A6 above, it will be appreciated that where x=1 in a given block copolymer arm, the linking moiety (LM) is present and the relevant parts of the branched polymer are indirectly covalently coupled to each other through the linking moiety (LM). Each of the at least 3 block co-polymer arms of the branched polymer must have present at least one linking moiety that is biodegradable (i.e. a biodegradable linking moiety).

In another embodiment, each of the at least three block co-polymer chains comprise two hydrophilic polymer blocks and a cationic polymer block, where the cationic polymer block is (i) located in between, and (ii) covalently coupled to, each of the two hydrophilic polymer blocks. In that case, the block copolymer chains may be conveniently referred to as having an A-B-A tri-block structure, where each A in a chain may be the same or different and represents a hydrophilic polymer block, and B represents the cationic polymer block.

Alternatively, each of the at least three block co-polymer chains may comprise a hydrophilic polymer block and two cationic polymer blocks, where the hydrophilic polymer block is (i) located in between, and (ii) covalently coupled to, each of the two cationic polymer blocks. In that case, the block copolymer chains may be conveniently referred to as having a B-A-B tri-block structure, where each B in a chain may be the same or different and represents a cationic polymer block, and A represents the hydrophilic polymer block.

In one embodiment, each of the at least three block co-polymer arms comprise two hydrophilic polymer blocks and a cationic polymer block, where the cationic polymer block is (i) located in between, and (ii) covalently coupled through a linking moiety to, each of the two hydrophilic polymer blocks.

Alternatively, each of the at least three block co-polymer arms may comprise a hydrophilic polymer block and two cationic polymer blocks, where the hydrophilic polymer block is (i) located in between, and (ii) covalently coupled through a linking moiety to, each of the two cationic polymer blocks.

In a further embodiment, each of the at least three block co-polymer arms comprise two hydrophilic polymer blocks and a cationic polymer block, where the cationic polymer block is (i) located in between, and (ii) covalently coupled through a biodegradable linking moiety to, each of the two hydrophilic polymer blocks.

In another embodiment, each of the at least three block co-polymer arms may comprise a hydrophilic polymer block and two cationic polymer blocks, where the hydrophilic polymer block is (i) located in between, and (ii) covalently coupled through a biodegradable linking moiety to, each of the two cationic polymer blocks.

According to such embodiments, the branched polymer may be conveniently represented by formulae (A7) and (A8) below:

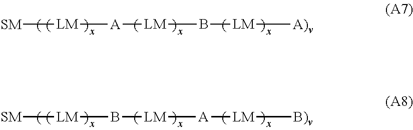

where SM represents the support moiety, LM represents a linking moiety, A represents a hydrophilic polymer block, B represents a cationic polymer block, each x is independently 0 or 1, and v is an integer greater than or equal to 3, such that (i) A, B, optionally together with LM, represent a block co-polymer arm of the branched polymer, and (ii) in each of the at least 3 block co-polymer arms at least one x=1 and the LM associated with that x=1 is a biodegradable linking moiety.

In structures A7 and A8 above, it will be appreciated that where x=0 in a given block copolymer arm, the linking moiety (LM) is not present and the relevant parts of the branched polymer are directly covalently coupled to each other.

In structures A7 and A8 above, it will be appreciated that where x=1 in a given block copolymer arm, the linking moiety (LM) is present and the relevant parts of the branched polymer are indirectly covalently coupled to each other through the linking moiety (LM). Each of the at least 3 block co-polymer arms of the branched polymer must have present at least one linking moiety that is biodegradable (i.e. a biodegradable linking moiety).

Where a block co-polymer arm comprises two hydrophilic polymer blocks or two cationic polymer blocks, each hydrophilic polymer block in the arm may be the same or different and each cationic polymer block in the arm may be the same or different.

In another embodiment, each of the at least three block co-polymer chains comprise a cationic polymer block that is covalently coupled to a hydrophobic polymer block, the hydrophobic polymer block itself being coupled to a hydrophilic polymer block. In that case, the block copolymer chains may be conveniently referred to as having a B-C-A tri-block structure, where B represents the cationic polymer block, C represents the hydrophobic polymer block, and A represents the hydrophilic polymer block.

Where each of the at least three block co-polymer arms comprise a cationic polymer block that is covalently coupled to a hydrophobic polymer block, the hydrophobic polymer block itself being coupled to a hydrophilic polymer block, the cationic polymer block of each chain may be covalently coupled to the support moiety, or the hydrophilic polymer block of each chain may be covalently coupled to the support moiety.

At least one of the linking moieties associated with each of the at least block copolymer chains is a biodegradable linking moiety

According to such embodiments, the branched polymer may be conveniently represented by formulae (A9) and (A10) below:

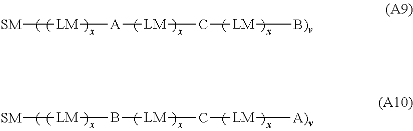

where SM represents the support moiety, LM represents a linking moiety, A represents a hydrophilic polymer block, B represents a cationic polymer block, C represents a further polymer block such as a hydrophobic polymer block, each x is independently 0 or 1, and v is an integer greater than or equal to 3, such that (i) A, B, and C, optionally together with LM, represent a block co-polymer arm of the branched polymer, and (ii) in each of the at least 3 block co-polymer arms at least one x=1 and the LM associated with that x=1 is a biodegradable linking moiety.

Those skilled in the art will appreciate that there may be other permutations and combinations of A, B. C and LM that can form a branched polymer according to the teaching outlined herein. For example, a block co-polymer chain may be in the form of a higher block copolymer, such as a tetra-, penta-, or a hexa- etc block copolymer.

Where each of the block copolymer chains comprise two or more linking moieties, each linking moiety may be the same or different.

By the block co-polymer chain comprising a “cationic polymer block” is meant it comprises a discernable block within the copolymer chain structure that presents or is capable of presenting a net positive charge.

By the block co-polymer chain comprising a “hydrophilic polymer block” is meant it comprises a discernable block within the copolymer chain structure that presents net hydrophilic character.

By the block co-polymer chain comprising a “further polymer block” is meant it comprises a discernable block within the copolymer chain structure that is not a cationic polymer block or a hydrophilic polymer block.

The further polymer block may be a hydrophobic polymer block. By the block co-polymer chain comprising a “hydrophobic polymer block” is meant it comprises a discernable block within the copolymer chain structure that presents net hydrophobic character.

Further detail regarding what is meant by the expressions “cationic polymer block”, “hydrophilic polymer block” and “hydrophobic polymer block” is presented below.

The block co-polymer that forms the arms of the branched polymer in accordance with the invention may be a linear block co-polymer.

Each polymer block in a block co-polymer arm of the branched polymer may be a homopolymer block or a copolymer block. Where a polymer block is a copolymer, the copolymer may be a gradient, a random or a statistical copolymer.

A given cationic polymer block and hydrophilic polymer block, and if present a further polymer block such as a hydrophobic polymer block, will generally each comprise the polymerised residues of a plurality of monomer units. Further detail concerning the monomers that may be used to form these blocks is presented below.

A cationic polymer block may comprise from about 5 to about 200, or about 40 to about 200, or about 80 to about 200 monomer residue units. Where a block co-polymer arm comprises two cationic polymer blocks, each cationic polymer block in the arm may independently comprise from about 5 to about 100, or about 20 to about 100, or about 40 to about 100 monomer residue units. Individually or collectively, the cationic polymer block(s) will present a net positive charge. Generally, at least about 10%, or at least 30%, or at least 40%, or at least 50%, or at least 70%, or at least 90%, or all of the monomer residue units that make up a cationic polymer block comprise a positive charge.

In one embodiment, a cationic polymer block comprises from about 5 to about 200, or about 40 to about 200, or about 80 to about 200 monomer residue units that each comprise positive charge.

Where a block copolymer chain comprises two cationic blocks, each cationic block may independently comprise from about 5 to about 100, or about 20 to about 100, or about 40 to about 100 monomer residue units that each comprise positive charge.

Where a branched polymer according to the invention is used in complex formation with a nucleic acid molecule, it will be appreciated that individually or collectively a cationic block(s) will comprise sufficient positive charge density to promote complexation with the nucleic acid molecule. Further detail in relation to such a complex formation embodiment is discussed below.

A hydrophilic polymer block may comprise from about 5 to about 200, or from about 30 to about 200, or from about 40 to about 180, or from about 50 to about 180, or from about 60 to about 180 monomer residue units. Where a block copolymer arm comprises two hydrophilic blocks, each hydrophilic block may independently comprise from about 5 to about 100, or about 15 to about 100, or about 20 to about 90, or from about 25 to about 90, or from about 30 to about 90 hydrophilic monomer residue units. Individually or collectively, the hydrophilic polymer block(s) will present net hydrophilic character. Generally, at least about 50%, or at least about 60%, or at least about 70%, or at least about 90%, or about 100% of the monomer residue units that form a hydrophilic polymer block will be hydrophilic monomer residue units.

In one embodiment, a hydrophilic polymer block comprises from about 5 to about 200, or from about 30 to about 200, or from about 40 to about 180, or from about 50 to about 180, or from about 60 to about 180 hydrophilic monomer residue units.

Where a block copolymer chain comprises two hydrophilic polymer blocks, each hydrophilic polymer block may independently comprise from about 5 to about 100, or about 15 to about 100, or about 20 to about 90, or from about 25 to about 90, or from about 30 to about 90 hydrophilic monomer residue units.

Where a block copolymer chain comprises a further polymer block, the further polymer block may comprise from about 5 to about 200, or from about 30 to about 200, or from about 40 to about 180, or from about 50 to about 180, or from about 60 to about 180 monomer residue units.

Where the further polymer block is a hydrophobic polymer block, the hydrophobic polymer block may comprise from about 5 to about 200, or from about 30 to about 200, or from about 40 to about 180, or from about 50 to about 180, or from about 60 to about 180 monomer residue units. The hydrophobic polymer block will present a net hydrophobic character. Generally, at least about 50%, or at least about 60%, or at least about 70%, or at least about 90%, or about 100% of the monomer residue units that form a hydrophobic polymer block will be hydrophobic monomer residue units.

In one embodiment, a hydrophobic polymer block comprises from about 5 to about 200, or from about 30 to about 200, or from about 40 to about 180, or from about 50 to about 180, or from about 60 to about 180 hydrophobic monomer residue units.

Terms such as hydrophilic and hydrophobic are generally used in the art to convey interactions between one component relative to another (e.g. attractive or repulsive interactions, or solubility characteristics) and not to quantitatively define properties of a particular component relative to another.

For example, a hydrophilic component is more likely to be wetted or solvated by an aqueous medium such as water, whereas a hydrophobic component is less likely to be wetted or solvated by an aqueous medium such as water.

In the context of the present invention, a hydrophilic polymer block is intended to mean a polymer block that exhibits solubility or miscibility in an aqueous medium, including biological fluids such as blood, plasma, serum, urine, saliva, milk, seminal fluid, vaginal fluid, synovial fluid, lymph fluid, amniotic fluid, sweat, and tears; as well as an aqueous solution produced by a plant, including, for example, exudates and guttation fluid, xylem, phloem, resin, and nectar.

In contrast, a hydrophobic polymer block is intended to mean a polymer block that exhibits little or no solubility or miscibility in an aqueous medium, including biological fluids such as blood, plasma, serum, urine, saliva, milk, seminal fluid, vaginal fluid, synovial fluid, lymph fluid, amniotic fluid, sweat, and tears; as well as an aqueous solution produced by a plant, including, for example, exudates and guttation fluid, xylem, phloem, resin, and nectar.

The hydrophilic polymer block(s) will generally be selected such that the branched polymer is rendered soluble or miscible in aqueous media.

The cationic polymer block(s) may also exhibit hydrophilic character such that it is soluble or miscible in aqueous media.

In one embodiment, the branched polymer does not comprise polymerised monomer residue units bearing negative charge. In other words, in one embodiment the branched polymer is not an ampholytic branched polymer.

Reference herein to “positive” or “negative” charge associated with a cationic polymer block or nucleic acid molecule, respectively, is intended to mean that the cationic polymer block, or nucleic acid molecule has one or more functional groups or moieties that present, or are intended to and are capable of presenting, a positive or negative charge, respectively.

Accordingly, such a functional group or moiety may inherently bear that charge, or it may be capable of being converted into a charged state, for example through addition or removal of an electrophile. In other words, in the case of a positive charge, a functional group or moiety may have an inherent charge such as a quaternary ammonium functional group or moiety, or a functional group or moiety per se may be neutral, yet be chargeable to form a cation through, for example, pH dependent formation of a tertiary ammonium cation, or quaternerisation of a tertiary amine group. In the case of negative charge, a functional group or moiety may, for example, comprise an organic acid salt that provides for the negative charge, or a functional group or moiety may comprise an organic acid which may be neutral, yet be chargeable to form an anion through, for example, pH dependent removal of an acidic proton.

In one embodiment, a cationic polymer block may be prepared using monomer that contains a functional group or moiety that is in a neutral state and can subsequently converted into a positively charged state. For example, the monomer may comprise a tertiary amine functional group, which upon being polymerised to form the cationic polymer block is subsequently quaternarised into a positively charged state.

Those skilled in the art will appreciate that in a charged state, a cation per se associated with a cationic polymer block, or an anion per se associated with, for example, a nucleic acid molecule, will have a suitable counter ion associated with it.

The number of monomer residue units that make up each block co-polymer chain will generally range from about 5 to about 500, or from about 10 to about 300, or from about 20 to about 150.

The branched polymer comprises at least three block co-polymer chains. In one embodiment, the branched polymer comprises from 3 to 12 block co-polymer chains, or from 3 to 9 block co-polymer chains, or from 3 to 6 block co-polymer chains.

For avoidance of any doubt, each block co-polymer chain of a given branched polymer according to the invention has substantially the same molecular composition.

Examples of branched polymers according to the invention may be illustrated with reference to general formulae A11-A13 below:

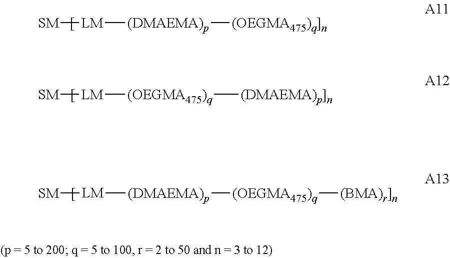

Where SM is a support moiety, LM is a biodegradable linking moiety, DMAEMA is a polymerised residue of 2-(N,N-dimethylamino)ethyl methacrylate. OEGMA is a polymerised residue of oligo(ethyleneglycol) methyl ether methacrylate and BMA is a polymerised residue of n-butyl methacrylate.

In one embodiment, the branched polymer further comprises a targeting ligand and/or an imaging agent. In that case a targeting ligand or an imaging agent will generally be covalently coupled to the branched polymer. A targeting ligand or an imaging agent may be covalently coupled to the support moiety, the block co-polymer chain of the branched polymer, or a combination thereof.

In one embodiment, the branched polymer may therefore be conveniently represented by formulae (A14)-(A16) below:

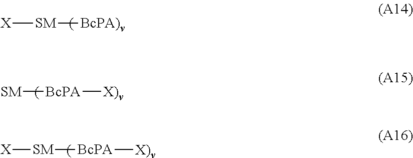

where SM represents the support moiety, BcPA represents a block co-polymer chain, X represents a targeting ligand or an imaging agent, in the case of formula (A16) each X may be the same or different, and v is an integer greater than or equal to 3.

Examples of suitable targeting ligands that may be coupled to the branched polymer include sugars and oligosaccharides derived from those sugars, peptides, proteins, aptamers, and cholesterol. Examples of suitable sugars include galactose, mannose, and glucosamine. Examples of suitable peptides include bobesin, lutanizing hormone releasing peptide, cell penetrating peptides (CPP's), GALA peptide, influenza-derived fusogeneic peptides, RGD peptide, poly(arginine), poly(lycine), penetratin, tat-peptide, and transportan. Other ligands such as folic acid that can target cancer cells may also be coupled to the branched polymer. Examples of suitable proteins include transferring protamine, and antibodies such as anti-EGFR antibody and anti-K-ras antibody.

Examples of suitable imaging agents that may be coupled to the branched polymer include Polyfluor™ (Methacryloxyethyl thiocarbamoyl rhodamine B), Alexa Fluor 568, and BOPIDY dye.

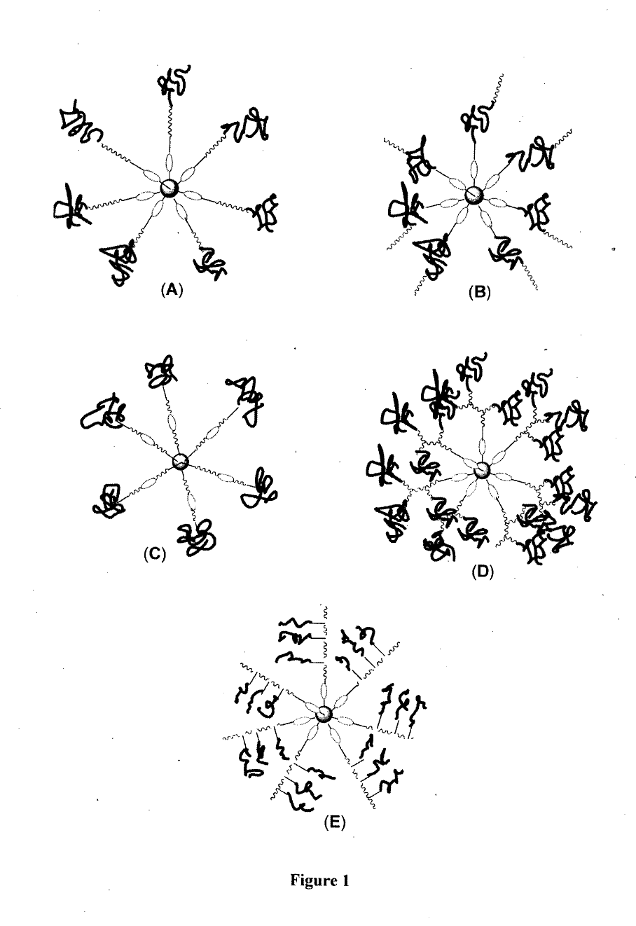

To further illustrate the nature of branched polymers in accordance with the invention, reference is made to FIG. 1 in which [symbol] represents the support moiety, ______ represents a general covalent bond, [symbol] represents a biodegradable covalent coupling or linking moiety, [symbol] represents a cationic polymer block, and [symbol] represents a hydrophilic polymer block. Structure (A) therefore illustrates a branched polymer where 6 linear block copolymer chains, each comprising a cationic polymer block covalently coupled to a hydrophilic polymer block, are coupled to a support moiety through a biodegradable covalent coupling. In this case, the cationic polymer block is coupled directly to the biodegradable covalent coupling. Structure (B) therefore illustrates a branched polymer where 6 linear block copolymer chains, each comprising a cationic polymer block covalently coupled to a hydrophilic polymer block, are coupled to a support moiety through a biodegradable covalent coupling. In this case, the hydrophilic polymer block is coupled directly to the biodegradable covalent coupling. Structure (C) therefore illustrates a branched polymer where 6 linear block copolymer chains, each comprising a cationic polymer block covalently coupled to a hydrophilic polymer block, are coupled to a support moiety. In this case, each chain has (i) a cationic polymer block coupled directly to the support moety, and (ii) two cationic polymer blocks directly coupled through a biodegradable covalent coupling. Structure (D) therefore illustrates a branched polymer where 6 branched block copolymer chains, each comprising a cationic polymer block covalently coupled to a hydrophilic polymer block, are coupled to a support moiety through a biodegradable covalent coupling. In this case, the cationic polymer block is coupled directly to the biodegradable covalent coupling. Structure (E) therefore illustrates a branched polymer where 6 branched block copolymer chains, each comprising a cationic polymer block covalently coupled to a hydrophilic polymer block, are coupled to a support moiety through a biodegradable covalent coupling. In this case, the cationic polymer block is coupled directly to the biodegradable covalent coupling.

The branched polymers may be prepared by any suitable means.

In one embodiment, the process of preparing the branched polymer comprises the polymerisation of ethylenically unsaturated monomers. Polymerisation of the ethylenically unsaturated monomers is preferably conducted using a living polymerisation technique.

Living polymerisation is generally considered in the art to be a form of chain polymerisation in which irreversible chain termination is substantially absent. An important feature of living polymerisation is that polymer chains will continue to grow while monomer and reaction conditions to support polymerisation are provided. Polymer chains prepared by living polymerisation can advantageously exhibit a well defined molecular architecture, a predetermined molecular weight and narrow molecular weight distribution or low polydispersity.

Examples of living polymerisation include ionic polymerisation and controlled radical polymerisation (CRP). Examples of CRP include, but are not limited to, iniferter polymerisation, stable free radical mediated polymerisation (SFRP), atom transfer radical polymerisation (ATRP), and reversible addition fragmentation chain transfer (RAFT) polymerisation.

Equipment, conditions, and reagents for performing living polymerisation are well known to those skilled in the art.

Where ethylenically unsaturated monomers are to be polymerised by a living polymerisation technique, it will generally be necessary to make use of a so-called living polymerisation agent. By “living polymerisation agent” is meant a compound that can participate in and control or mediate the living polymerisation of one or more ethylenically unsaturated monomers so as to form a living polymer chain (i.e. a polymer chain that has been formed according to a living polymerisation technique).

Living polymerisation agents include, but are not limited to, those which promote a living polymerisation technique selected from ionic polymerisation and CRP.

In one embodiment of the invention, the branched polymer is prepared using ionic polymerisation.

In one embodiment of the invention, the branched polymer is prepared using CRP.

In a further embodiment of the invention, the branched polymer is prepared using iniferter polymerisation.

In another embodiment of the invention, the branched polymer is prepared using SFRP.

In a further embodiment of the invention, the branched polymer is prepared using ATRP.

In yet a further embodiment of the invention, the branched polymer is prepared using RAFT polymerisation.

A polymer formed by RAFT polymerisation may conveniently be referred to as a RAFT polymer. By virtue of the mechanism of polymerisation, such polymers will comprise residue of the RAFT agent that facilitated polymerisation of the monomer.

RAFT agents suitable for use in accordance with the invention comprise a thiocarbonylthio group (which is a divalent moiety represented by: —C(S)S—). RAFT polymerisation and RAFT agents are described in numerous publications such as WO 98/01478, Moad G.; Rizzardo, E; Thang S, H. Polymer 2008, 49, 1079-1131 and Aust. J. Chem., 2005, 58, 379-410; Aust. J. Chem., 2006, 59, 669-692; and Aust. J. Chem. 2009, 62, 1402-1472 (the entire contents of which are incorporated herein by reference). Suitable RAFT agents for use in preparing the branched polymers include xanthate, dithioester, dithiocarbamate and trithiocarbonate compounds.

RAFT agents suitable for use in accordance with the invention also include those represented by general formula (I) or (II):

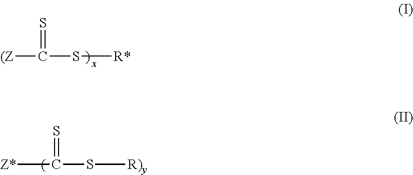

where Z and R are groups, and R* and Z* are x-valent and y-valent groups, respectively, that are independently selected such that the agent can function as a RAFT agent in the polymerisation of one or more ethylenically unsaturated monomers; x is an integer≧1; and y is an integer≧2.

In one embodiment, x is an integer≧3; and y is an integer≧3. In that case. R* and Z* may represent a support moiety (SM).

In order to function as a RAFT agent in the polymerisation of one or more ethylenically unsaturated monomers, those skilled in the art will appreciate that R and R* will typically be an optionally substituted organic group that function as a free radical leaving group under the polymerisation conditions employed and yet, as a free radical leaving group, retain the ability to reinitiate polymerisation. Those skilled in the art will also appreciate that Z and Z* will typically be an optionally substituted organic group that function to give a suitably high reactivity of the C═S moiety in the RAFT agent towards free radical addition without slowing the rate of fragmentation of the RAFT-adduct radical to the extent that polymerisation is unduly retarded.

In formula (I), R* is a x-valent group, with x being an integer≧1. Accordingly, R* may be mono-valent, di-valent, tri-valent or of higher valency. For example, R* may be a C₂₀ alkyl chain, with the remainder of the RAFT agent depicted in formula (I) presented as multiple substituent groups pendant from the chain. Generally, x will be an integer ranging from 1 to about 20, for example from about 2 to about 10, or from 1 to about 5. In one embodiment, x=2.

Similarly, in formula (II), Z* is a y-valent group, with y being an integer≧2. Accordingly, Z* may be di-valent, tri-valent or of higher valency. Generally, y will be an integer ranging from 2 to about 20, for example from about 2 to about 10, or from 2 to about 5.

Examples of R in RAFT agents used in accordance with the invention include optionally substituted, and in the case of R* in RAFT agents used in accordance with the invention include a x-valent form of optionally substituted, alkyl, alkenyl, alkynyl, aryl, acyl, carbocyclyl, heterocyclyl, heteroaryl, alkylthio, alkenylthio, alkynylthio, arylthio, acylthio, carbocyclylthio, heterocyclylthio, heteroarylthio, alkylalkenyl, alkylalkynyl, alkylaryl, alkylacyl, alkylcarbocyclyl, alkylheterocyclyl, alkylheteroaryl, alkyloxyalkyl, alkenyloxyalkyl, alkynyloxyalkyl, aryloxyalkyl, alkylacyloxy, alkylcarbocyclyloxy, alkyl heterocyclyloxy, alkylheteroaryloxy, alkylthioalkyl, alkenylthioalkyl, alkynylthioalkyl, arylthioalkyl, alkylacylthio, alkylcarbocyclylthio, alkylheterocyclylthio, alkylheteroarylthio, alkylalkenylalkyl, alkylalkynylalkyl, alkylarylalkyl, alkylacylalkyl, arylalkylaryl, arylalkenylaryl, arylalkynylaryl, arylacylaryl, arylacyl, arylcarbocyclyl, arylheterocyclyl, arylheteroaryl, alkenyloxyaryl, alkynyloxyaryl, aryloxyaryl, alkylthioaryl, alkenylthioaryl, alkynylthioaryl, arylthioaryl, arylacylthio, arylcarbocyclylthio, arylheterocyclylthio, arylheteroarylthio, and a polymer chain.

For avoidance of any doubt reference herein to “optionally substituted”, alkyl, alkenyl etc, is intended to mean each group such as alkyl and alkenyl is optionally substituted.

Examples of R in RAFT agents used in accordance with the invention also include optionally substituted, and in the case of R* in RAFT agents used in accordance with the invention also include an x-valent form of optionally substituted, alkyl; saturated, unsaturated or aromatic carbocyclic or heterocyclic ring; alkylthio; dialkylamino; an organometallic species; and a polymer chain.

Living polymerisation agents that comprise a polymer chain are commonly referred to in the art as “macro” living polymerisation agents. Such “macro” living polymerisation agents may conveniently be prepared by polymerising one or more ethylenically unsaturated monomers under the control of a given living polymerisation agent.

In one embodiment, such a polymer chain is formed by polymerising ethylenically unsaturated monomer under the control of a RAFT agent.

Examples of Z in RAFT agents used in accordance with the invention include optionally substituted, and in the case of Z* in RAFT agents used in accordance with the invention include a y-valent form of optionally substituted: F, Cl, Br, I, alkyl, aryl, acyl, amino, carbocyclyl, heterocyclyl, heteroaryl, alkyloxy, aryloxy, acyloxy, acylamino, carbocyclyloxy, heterocyclyloxy, heteroaryloxy, alkylthio, arylthio, acylthio, carbocyclylthio, heterocyclylthio, heteroarylthio, alkylaryl, alkylacyl, alkylcarbocyclyl, alkylheterocyclyl, alkylheteroaryl, alkyloxy alkyl, aryloxyalkyl, alkylacyloxy, alkylcarbocyclyloxy, alkylheterocyclyloxy, alkylheteroaryloxy, alkylthioalkyl, arylthioalkyl, alkylacylthio, alkylcarbocyclylthio, alkylheterocyclylthio, alkylheteroarylthio, alkylarylalkyl, alkylacylalkyl, arylalkylaryl, arylacylaryl, arylacyl, arylcarbocyclyl, arylheterocyclyl, arylheteroaryl, aryloxyaryl, arylacyloxy, arylcarbocyclyloxy, arylheterocyclyloxy, arylheteroaryloxy, alkylthioaryl, arylthioaryl, arylacylthio, arylcarbocyclylthio, arylheterocyclylthio, arylheteroarylthio, dialkyloxy-, diheterocyclyloxy- or diaryloxy-phosphinyl, dialkyl-, diheterocyclyl- or diaryl-phosphinyl, cyano (i.e. —CN), and —S—R, where R is as defined in respect of formula (II).

In one embodiment, the RAFT agent used in accordance with the invention is a trithiocarbonate RAFT agent and Z or Z* is an optionally substituted alkylthio group.

MacroRAFT agents suitable for use in accordance with the invention may obtained commercially, for example see those described in the SigmaAldrich catalogue (www.sigmaaldrich.com).

Other RAFT agents that can be used in accordance with the invention include those described in WO2010/083569 and Benaglia et al, Macromolecules. (42), 9384-9386, 2009, (the entire contents of which are incorporated herein by reference).

In one embodiment, the at least three block co-polymer arms of the branched polymer are formed using RAFT polymerisation.

In the lists herein defining groups from which. Z, Z*, R and R* may be selected, each alkyl, alkenyl, alkynyl, aryl, carbocyclyl, heteroaryl, heterocyclyl, and polymer chain moiety may be optionally substituted.

In the lists herein defining groups from which Z, Z*, R and R* may be selected, where a given Z, Z*, R or R* contains two or more subgroups (e.g. [group A][group B]), the order of the subgroups is not intended to be limited to the order in which they are presented (e.g. alkylaryl may also be considered as a reference to arylalkyl).

The Z, Z*. R or R* may be branched and/or optionally substituted. Where the Z, Z*, R or R* comprises an optionally substituted alkyl moiety, an optional substituent includes where a —CH₂— group in the alkyl chain is replaced by a group selected from —O—, —S—, —NR^{a}—, —C(O)— (i.e. carbonyl), —C(O)O— (i.e. ester), and —C(O)NR^{a}— (i.e. amide), where R^{a} may be selected from hydrogen, alkyl, alkenyl, alkynyl, aryl, carbocyclyl, heteroaryl, heterocyclyl, arylalkyl, and acyl.

Reference herein to a x-valent, y-valent, multi-valent or di-valent “form of . . . ” is intended to mean that the specified group is a x-valent, y-valent, multi-valent or di-valent radical, respectively. For example, where x or y is 2, the specified group is intended to be a divalent radical. Those skilled in the art will appreciate how to apply this rationale in providing for higher valent forms.

Preparation of the branched polymers will generally involve the polymerisation of ethylenically unsaturated monomers. Factors that determine copolymerisability of ethylenically unsaturated monomers are well documented in the art. For example, see: Greenlee, R. Z., in Polymer Handbook 3^{rd} edition (Brandup, J, and Immergut. E. H. Eds) Wiley: New York, 1989, p II/53 (the entire contents of which are incorporated herein by reference).

Suitable examples of ethylenically unsaturated monomers that may be used to prepare the branched polymers include those of formula (III):

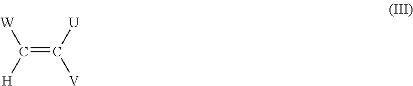

where U and W are independently selected from —CO₂H, —CO₂R¹, —COR¹, —CSR¹, —CSOR¹, —COSR¹, —CONH₂, —CONHR¹, —CONR¹₂, hydrogen, halogen and optionally substituted. C₁-C₄ alkyl or U and W form together a lactone, anhydride or imide ring that may itself be optionally substituted, where the optional substituents are independently selected from hydroxy, —CO₂H, —CO₂R¹, —COR¹, —CSR¹, —CSOR¹, —COSR¹, —CN, —CONH₂, —CONHR¹, —CONR¹₂, —OR¹, —SR¹, —O₂CR¹, —SCOR¹, and —OCSR¹;

V is selected from hydrogen. R¹, —CO₂H, —CO₂R¹, —COR¹, —CSR¹, —CSOR¹, —COSR¹, —CONH₂, —CONHR¹, —CONR¹₂, —OR¹, —SR¹, —O₂CR¹, —SCOR¹, and —OCSR¹;

where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain.

Specific examples of monomers of formula (III) include those outlined in one or more of WO 2010/083569, WO 98/01478, Moad. G.; Rizzardo, E; Thang S, H. Polymer 2008, 49, 1079-1131 and Aust. J. Chem. 2005, 58, 379-410; Aust. J. Chem., 2006, 59, 669-692; Aust. J. Chem., 2009, 62, 1402-1472. Greenlee, R. Z., in Polymer Handbook 3^{rd} edition (Brandup. J, and Immergut. E. H. Eds) Wiley: New York, 1989, p II/53 and Benaglia et al, Macromolecules. (42), 9384-9386, 2009 (the entire contents of which are incorporated herein by reference).

When discussing the types of monomers that may be used to prepare the branched polymer, it may be convenient to refer to the monomers as being hydrophilic, hydrophobic or cationic in character. By being hydrophilic, hydrophobic or cationic “in character” in this context is meant that upon polymerisation such monomers respectively give rise (directly or indirectly) to the hydrophilic, hydrophobic and cationic polymer blocks that form the block co-polymer arms. For example, a hydrophilic polymer block that forms part of a block co-polymer arm will generally be prepared by polymerising a monomer composition that comprises hydrophilic monomer.

As a guide only, examples of hydrophilic ethylenically unsaturated monomers include, but are not limited to, acrylic acid, methacrylic acid, hydroxyethyl methacrylate, hydroxypropyl methacrylate, oligo(alkylene glycol)methyl ether (meth)acrylate (OAG(M)A), acrylamide and methacrylamide, hydroxyethyl acrylate, N-methylacrylamide, N,N-dimethylacrylamide and N,N-di methylaminoethyl methacrylate, N,N-dimethylaminopropyl methacrylamide, N-hydroxypropyl methacrylamide, 4-acryloylmorpholine, 2-acrylamido-2-methyl-1-propanesulfonic acid, phosphorylcholine methacrylate and N-vinyl pyrolidone.

Where the monomer used gives rise to a cationic polymer block, as previously outlined, the so formed polymer block may not inherently be in a charged cationic state. In other words, the polymer block may need to be reacted with one or more other compounds to be converted into a charged cationic state. For example, the monomer selected to form a cationic polymer block may comprise a tertiary amine functional group. Upon polymerising the monomer to form the cationic polymer block, the tertiary amine functional group can be subsequently quaternarised into a positively charged state.

As a guide only, examples of cationic ethylenically unsaturated monomers include, but are not limited to, N,N-dimethyaminoethyl methacrylate, N,N-diethylaminoethyl methacrylate, N,N-dimethylaminoethyl acrylate, N,N-diethylaminoethyl acrylate, 2-aminoethyl methacrylate hydrochloride, N-[3-(N,N-dimethylamino)propyl]methacrylamide, N-(3-aminopropyl)methacrylamide hydrochloride, N-[3-(N,N-dimethylamino)propyl]acrylamide, N-[2-(N,N-dimethylamino)ethyl]methacrylamide, 2-N-morpholinoethyl acrylate, 2-N-morpholinoethyl methacrylate, 2-(N,N-dimethylamino)ethyl acrylate, 2-(N,N-dimethylamino)ethyl methacrylate, 2-(N,N-diethylamino)ethyl methacrylate, 2-acryloxyyethyltrimethylammonium chloride, mthacrylamidopropyltrimethylammonium chloride, 2-(tert-butylamino)ethyl methacrylate, allyldimethylammonium chloride, 2-(dethylamino)ethylstyrene, 2-vinylpyridine, and 4-vinylpyridine.

As a guide only, examples of hydrophobic ethylenically unsaturated monomers include, but are not limited to, styrene, alpha-methyl styrene, butyl acrylate, butyl methacrylate, amyl methacrylate, hexyl methacrylate, lauryl methacrylate, stearyl methacrylate, ethyl hexyl methacrylate, crotyl methacrylate, cinnamyl methacrylate, oleyl methacrylate, ricinoleyl methacrylate, cholesteryl methacrylates, cholesteryl acrylate, vinyl butyrate, vinyl tert-butyrate, vinyl stearate and vinyl laurate.

In the case of the hydrophilic ethylenically unsaturated monomer OAG(M)A, the alkylene moiety will generally be a C₂-C₆, for example a C₂ or C₃, alkylene moiety. Those skilled in the art will appreciate that the “oligo” nomenclature associated with the “(alkylene glycol)” refers to the presence of a plurality of alkylene glycol units. Generally, the oligo component of the OAG(M)A will comprise about 2 to about 200, for example from about 2 to about 100, or from about 2 to about 50 or from about 2 to about 20 alkylene glycol repeat units.

The hydrophilic polymer block of the block co-polymer arm may therefore be described as comprising the polymerised residues of hydrophilic ethylenically unsaturated monomers.

The cationic polymer block of the block co-polymer arm may therefore be described as comprising the polymerised residues of cationic ethylenically unsaturated monomers.

The hydrophobic polymer block of the block co-polymer arm may therefore be described as comprising the polymerised residues of hydrophobic ethylenically unsaturated monomers.

Where a free radical polymerisation technique is to be used in polymerising one or more ethylenically unsaturated monomers so as to form at least part of the block co-polymer arms, the polymerisation will usually require initiation from a source of free radicals.

A source of initiating radicals can be provided by any suitable means of generating free radicals, such as the thermally induced homolytic scission of suitable compound(s) (thermal initiators such as peroxides, peroxyesters, or azo compounds), the spontaneous generation from monomers (e.g. styrene), redox initiating systems, photochemical initiating systems or high energy radiation such as electron beam, X- or gamma-radiation. Examples of such initiaiators may be found in, for example, WO 2010/083569 and Moad and Solomon “The Chemistry of Free Radical Polymerisation”, Pergamon, London, 1995, pp 53-95 (the entire contents of which are incorporated herein by reference).

The branched polymer may be constructed using techniques know in the art. For example, the block co-polymer arms of the polymer may be first formed using an appropriate polymerisation reaction and then subsequently coupled to a suitable support moiety. This technique is known as a “coupling onto” approach.

Alternatively, the block co-polymer arms of the polymer may formed by polymerising monomers directly from a suitable support moiety. This technique is known as a “core first” approach.

It may also be possible to use a combination of coupling onto and core first approaches. For example, monomer may be polymerised directly from a suitable support moiety to form the cationic polymer block (core first). A preformed hydrophilic polymer block may then be coupled to the cationic polymer block to form the block co-polymer arms (coupling onto).

Where a core first approach is employed, in one embodiment the branched polymer may be prepared using a living polymerisation agent of general formula (IV):

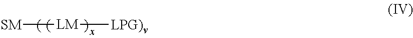

where SM represents the support moiety, LM represents a linking moiety (if present), LPG represents a living polymerisation group, x is 0 or 1, and v is an integer greater than or equal to 3.

In one embodiment, LPG is selected from a group that promotes living ionic polymerisation or controlled radical polymerisation. Where LPG promotes controlled radical polymerisation it may be conveniently represented as CRPG.

In one embodiment, CRPG is selected from a group that promotes iniferter polymerisation, SFRP polymerisation, ATRP, or RAFT polymerisation.

Where CRPG promotes RAFT polymerisation, formula (IV) may be conveniently represented by formula (V):

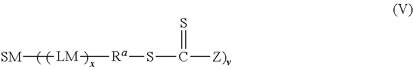

where SM represents the support moiety, LM represents a linking moiety (if present), R^{a} represents a divalent form of R* as herein defined in respect of formula (I), Z is as herein defined in respect of formula (I), x is 0 or 1, and v is an integer greater than or equal to 3. It will be appreciated that LM and R^{a} in general formula (V) can together function to couple the active RAFT moiety (i.e. S—C(S)—Z) to the support moiety SM. The function of LM in general formula (V) is therefore identify a feature of the molecular structure that may or may not be present, and if present may or may not be biodegradable. R^{a} in general formula (V) may also be biodegradable, but this function is not essential. In contrast. LM (when present) is presented in general formula (V) specifically for the option of it being biodegradable.

In one embodiment, the features of general formula (V) are each independently defined by: SM which is selected from alkyl, aryl, heterocyclyl, heteroaryl, and a coordination complex; LM which is biodegradable through one or more functional groups selected from ester, anhydride, carbonate, peroxide, peroxyester, phosphate, thioester, urea, thiourethane, ether, disulfide, carbamate (urethane) and boronate ester; R^{a} which is selected from a divalent form of optionally substituted alkyl, aryl, heterocyclyl, heteroaryl (where preferred optional substituents include those defined herein and in particular alkyl and cyano); and Z which is selected from optionally substituted alkyl, aryl, alkylthio, and arylthio (where preferred optional substituents include those defined herein and in particular alkyl and cyano).

It will be appreciated that by LM being “biodegradable through one or more functional groups” means that such functional groups directly form part of the string of atoms that provide the covalent coupling. In other words, at least one atom of such functional groups is present in the direct string of atoms that covalently couple the relevant sections of the polymer (e.g. the support moiety to the block co-polymer chains).

In another embodiment, general formula (V) has a structure (Va) or (Vb):

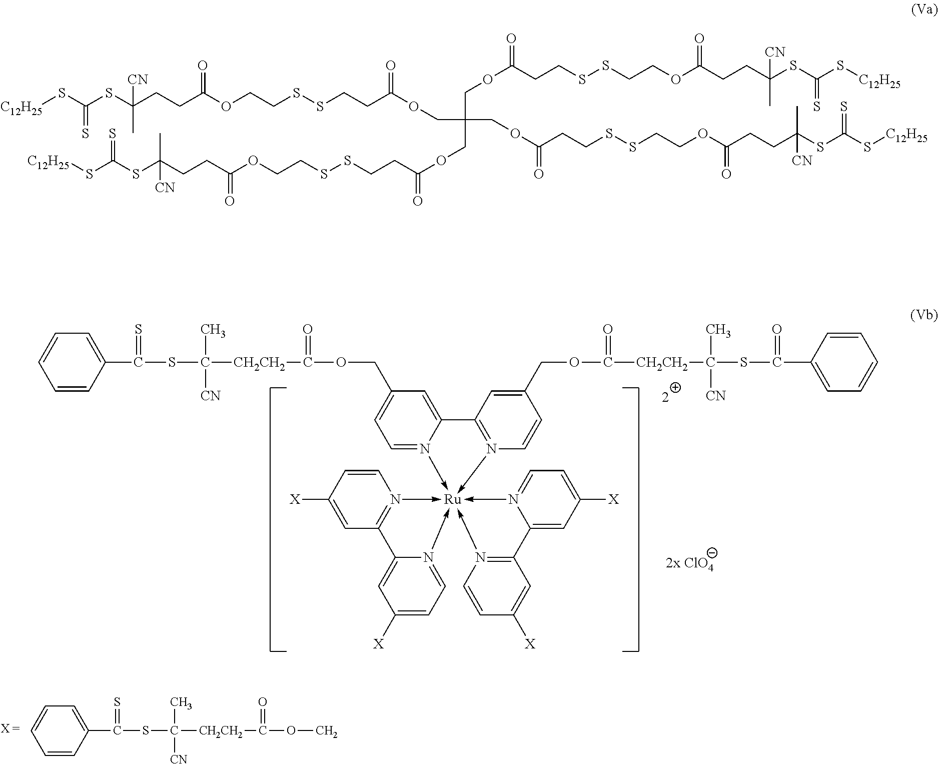

The present invention also provides a complex comprising the branched polymer and a nucleic acid molecule. The term “complex” as used herein refers to the association by ionic bonding of the branched polymer and the nucleic acid molecule. The ionic bonding is derived through electrostatic attraction between oppositely charged ions associated with the cationic polymer block(s) of the branched polymer and the nucleic acid molecule. It will be appreciated that the cationic polymer block will provide for positive charge, and accordingly the nucleic acid molecule will provide for negative charge so as to promote the required electrostatic attraction and formation of the complex.

The net negative charge on the nucleic acid molecule will generally be derived from the negatively charged nucleic acids per se (e.g. from the phosphate groups). Any modification(s) made to the nucleic acid molecule should retain a net negative charge to the extent that it allows formation of a complex through ionic bonding with the branched polymer.

Without wishing to be limited by theory, the branched polymer and nucleic acid molecule are believed to form nanoparticles through ionic interactions between the negatively charged backbone of the nucleic acid molecule and the cationic block of the branched polymer. Depending on the number of cationic charges in a given branched polymer, one or more nucleic acid molecules may associate with the polymer to form complexes, and the number of the complexed nucleic acid molecules may increase with the increasing number of arms/branches in the polymer. Accordingly, a branched polymer may have advantages in that more nucleic acid molecules can be complexed per branched polymer molecule than their linear counterparts. Furthermore, branched polymers, due to the presence of multiple cationic blocks within the each polymer molecule, may enable the formation of large complex structures with nucleic acid molecules acting as bridging molecules between two or more branched polymer molecules.

The complex comprising the branched polymer and nucleic acid molecule may be prepared using known techniques for preparing cationic polymer/nucleic acid molecule complexes. For example, a required amount of polymer suspended in water may be introduced to a Container comprising reduced serum media such as Opti-MEM®. The required amount of nucleic acid molecule may then be introduced to this solution and the resulting mixture vortexed for an appropriate amount of time so as to form the complex.

The nucleic acid molecule may be obtained commercially or prepared or isolated using techniques well known in the art.

There is no particular limitation concerning the ratio of nucleic acid molecule to branched polymer that may be used to form the complex. Those skilled in the art will appreciate that charge density (as indicated by zeta potential) of the branched polymer and nucleic acid, molecule, together with the ratio of branched polymer and nucleic acid molecule, will effect the overall charge/neutral state of the resulting complex.

In one embodiment, the complex has a positive Zeta potential. In a further embodiment, the complex has a positive Zeta potential ranging from greater than 0 mV to about 50 mV, for example from about 10 mV to about 40 mV, or from about 15 mV to about 30 mV, or from about 20 mV to about 25 mV.

The Zeta potential of a complex in accordance with the present invention is that as measured by Malvern Zetasizer. The Zeta potential is calculated from the measurement of the mobility of particles (electrophoertic mobility) in an electrical field and the particle size distribution in the sample.

The term “nucleic acid molecule” used herein refers to nucleic acid molecules including DNA (gDNA, cDNA), oligonucleotides (double or single stranded), RNA (sense RNAs, antisense RNAs, mRNAs, tRNAs, rRNAs, small interfering RNAs (siRNAs), double-stranded RNAs (dsRNA), short hairpin RNAs (shRNAs), piwi-interacting RNAs (PiRNA), micro RNAs (miRNAs), small nucleolar RNAs (SnoRNAs), small nuclear (SnRNAs) ribozymes, aptamers, DNAzymes, ribonuclease-type complexes and other such molecules as herein described. For the avoidance of doubt, the term “nucleic acid molecule” includes non-naturally occurring modified forms, as well as naturally occurring forms.

In some embodiments, the nucleic acid molecule comprises from about 8 to about 80 nucleobases (i.e. from about 8 to about 80 consecutively linked nucleic acids). One of ordinary skill in the art will appreciate that the present invention embodies nucleic acid molecules of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length.

The term “nucleic acid molecule” also includes other families of compounds such as oligonucleotide analogs, chimeric, hybrid and mimetic forms.

Chimeric oligomeric compounds may also be formed as composite structures of two or more nucleic acid molecules, including, but not limited to, oligonucleotides, oligonucleotide analogs, oligonucleotides and oligonucleotide mimetics. Routinely used chimeric compounds include but are not limited to hybrids, hemimers, gapmers, extended gapmers, inverted gapmers and blockmers, wherein the various point modifications and or regions are selected from native or modified DNA and RNA type units and/or mimetic type subunits such as, for example, locked nucleic acids (LNA), peptide nucleic acids (PNA), morpholinos, and others. The preparation of such hybrid structures is described for example in U.S. Pat. Nos. 5,013,830; 5,149,797; 5,220.007; 5,256,775; 5,366,878; 5,403,711; 5,491,133; 5,565,350; 5,623,065; 5,652,355; 5,652,356; and 5,700,922, each of which is herein incorporated by reference in its entirety.

RNA and DNA aptamers are also contemplated. Aptamers are nucleic acid molecules having specific binding affinity to non-nucleic acid or nucleic acid molecules through interactions other than classic Watson-Crick base pairing. Aptamers are described, for example, in U.S. Pat. Nos. 5,475,096; 5,270,163; 5,589,332; 5,589,332; and 5,741,679. An increasing number of DNA and RNA aptamers that recognize their non-nucleic acid targets have been developed and have been characterized (see, for example, Gold et al., Annu. Rev. Biochem., 64: 763-797.1995; Bacher et al., Drug Discovery Today, 3(6): 265-273, 1998).

Further modifications can be made to the nucleic acid molecules and may include conjugate groups attached to one of the termini, selected nucleobase positions, sugar positions or to one of the internucleoside linkages.

The present invention also provides a method of delivering a nucleic acid molecule to a cell, the method comprising:

(a) providing a complex comprising a branched polymer and a nucleic acid molecule, the branched polymer comprising a support moiety and at least three block co-polymer chains covalently coupled to and extending from the moiety, wherein:
(i) each of the at least three block co-polymer chains comprise (a) a cationic polymer block that is covalently coupled to a hydrophilic polymer block, or (b) a cationic polymer block that is covalently coupled to a hydrophobic polymer block, said hydrophobic polymer block being covalently coupled to a hydrophilic polymer block; and
(ii) at least one of said covalent couplings associated with each of said block co-polymer chains is biodegradable; and
   (b) delivering the complex to the cell.

This method may be performed in vivo, ex vivo or in vitro.

The present invention further provides a method of gene therapy comprising the administration to a subject in need thereof a therapeutically effective amount of the nucleic acid molecule complex according to the present invention, as herein described.

The relevance of DNA repair and mediated recombination as gene therapy is apparent when studied, for example, in the context of genetic diseases such as cystic fibrosis, hemophilia and globinopathies such as sickle cell anemia and beta-thalassemia. For example, if the target gene contains a mutation that is the cause of a genetic disorder, then delivering a nucleic acid molecule into the cell(s) of a subject can be useful for facilitating mutagenic repair to restore the DNA sequence of the abnormal target gene to normal. Alternatively, the nucleic acid molecule introduced to the cell(s) of a subject may lead to the expression of a gene that is otherwise suppressed or silent in the disease state. Such nucleic acid molecules may themselves encode the silent or suppressed gene, or they may activate transcription and/or translation of an otherwise suppressed or silent target gene.

It would be understood by those skilled in the art that the disease or condition to be treated using the method of the present invention may be any disease or condition capable of treatment by gene therapy and the choice of the genetic material (i.e., nucleic acid molecule) to be used will clearly depend upon the particular disease or condition. Diseases or conditions that may be treated include, but are not limited to, cancers (e.g. myeloid disorders), thalassemia, cystic fibrosis, deafness, vision disorders (e.g. Leber's congenital amaurosis), diabetes, Huntingdon's disease, X-linked severe combined immunodeficiency disease and heart disease. Alternatively, the gene therapy may be used to introduce non-endogenous genes, for example, genes for bioluminescence, or to introduce genes which will knock out endogenous genes (e.g. RNA interference).

It would also be understood by those skilled in the art that the nature of the nucleic acid molecule will invariably depend on the disease or condition to be treated or prevented. For example, a disease or condition that is attributed, at least in part, to an accumulation of fibrotic extracellular matrix material (e.g., type II collagen), can be treated or prevented by delivering the nucleic acid molecule complex of the present invention to the subject (in a targeted or non-targeted approach), wherein the nucleic acid molecule (e.g., siRNA) is capable of silencing the gene that encodes the extracellular matrix material. In some embodiments, the disease or condition is an infectious disease, an inflammatory disease, or a cancer.

Where delivery of the nucleic acid molecule complex to a cell in accordance with the present invention is performed in vivo, the nucleic acid molecule complex can be introduced to the cell by any route of administration that is appropriate under the circumstances. For instance, where systemic delivery is intended, the complex may be administered intravenously, subcutaneously, intramuscularly, orally, etc. Alternatively, the complex may be targeted to a particular cell or cell type by means known to those skilled in the art. Targeting may be desirable for a variety of reasons such as, for example, to target cancer cells if the nucleic acid molecule is unacceptably toxic to non-cancerous cells or if it would otherwise require too high a dosage. Targeted delivery may be achieved by any means know to those skilled in the art including, but not limited to, receptor-mediated targeting or by administering the nucleic acid complex directly to the tissue comprising the target cell(s).

Receptor-mediated targeting may be achieved, for example, by conjugating the nucleic acid molecule to a protein ligand, e.g., via polylysine. Ligands are typically chosen on the basis of the presence of the corresponding ligand receptors on the surface of the target cell/tissue type. These ligand-nucleic acid molecule conjugates can be complexed with a branched polymer in accordance with the present invention and administered systemically if desired (e.g., intravenously), where they will be directed to the target cell/tissue where receptor binding occurs.

In one embodiment, the method of delivering a nucleic acid molecule to a cell in accordance with the present invention is performed ex vivo. For example, cells are isolated from the subject and introduced ex vivo with the nucleic acid molecule complex of the present invention to produce cells comprising the exogenous nucleic acid molecule. The cells may be isolated from the subject to be treated or from a syngeneic host. The cells are then reintroduced back into the subject (or into a syngeneic recipient) for the purpose of treatment or prophyaxis. In some embodiments, the cells can be hematopoietic progenitor or stem cells.

In one embodiment, the nucleic acid, molecule is delivered to a cell for the purpose of silencing (or suppressing) gene expression. In some embodiments, gene expression is silenced by reducing translational efficiency or reducing message stability or a combination of these effects. In some embodiments, splicing of the unprocessed RNA is the target goal leading to the production of non-functional or less active protein.

In some embodiments, gene expression is silenced by delivering to a cell a DNA molecule, including but not limited to, gDNA, cDNA and DNA oligonucleotides (double or single stranded).

In some embodiments, gene expression is silenced by RNA interference (RNAi). Without limiting the present invention to a particular theory or mode of action, “RNA interference” typically describes a mechanism of silencing gene expression that is based on degrading or otherwise preventing the translation of mRNA, for example, in a sequence specific manner. It would be understood by those skilled in the art that the exogenous interfering RNA molecules may lead to either mRNA degradation or mRNA translation repression. In some embodiments, RNA interference is achieved by altering the reading frame to introduce one or more premature stop codons that lead to non-sense mediated decay. RNAi includes the process of gene silencing involving double stranded (sense and antisense) RNA that leads to sequence specific reduction in gene expression via target mRNA degradation. RNAi is typically mediated by short double stranded siRNAs or single stranded microRNAs (miRNA). In some embodiments, RNAi is initiated when a strand of RNA from either of these molecules forms a complex referred to as an RNA-induced silencing complex (RISC) which targets complementary RNA and suppresses translation. The process can be exploited for research purposes and for therapeutic application (see for example, Izquierdo et al., Cancer Gene Therapy, 12(3): 217-27, 2005).

Other oligonucleotides having RNA-like properties have also been described and many more different types of RNAi may be developed. For example, antisense oligonucleotides have been used to alter exon usage and to modulate pre-RNA splicing (see, for example, Madocsai et al., Molecular Therapy, 12: 1013-1022, 2005 and Aartsma-Rus et al., BMC Med. Genet., 8: 43, 2007). Antisense and iRNA compounds may be double stranded or single stranded oligonucleotides which are RNA or RNA-like or DNA or DNA-like molecules that hybridize specifically to DNA or RNA of the target gene of interest.

Examples of RNA molecules suitable for use in the context of the present invention include, but are not limited to:
(i) long double stranded RNA (dsRNA)—these are generally produced as a result of the hybridisation of a sense RNA strand and an antisense RNA strand which are each separately transcribed by their own vector. Such double stranded molecules are typically not characterised by a hairpin loop. These molecules are required to be cleaved by an enzyme such as Dicer in order to generate short interfering RNA (siRNA) duplexes. This cleavage event preferably occurs in the cell in which the dsRNA is transcribed.
(ii) hairpin double stranded RNA (hairpin dsRNA)—these molecules exhibit a stem-loop configuration and are generally the result of the transcription of a construct with inverted repeat sequences which are separated by a nucleotide spacer region, such as an intron. These molecules are generally of longer RNA molecules which require both the hairpin loop to be cleaved off and the resultant linear double stranded molecules to be cleaved by the enzyme Dicer in order to generate siRNA. This type of molecule has the advantage of being expressible by a single vector.
(iii) short interfering RNA (siRNA)—these can be synthetically generated or, recombinantly expressed by the promoter based expression of a vector comprising tandem sense and antisense strands each characterised by its own promoter and a 4-5 thymidine transcription termination site. This enables the generation of two separate transcripts which subsequently anneal. In some embodiments, these transcripts may be of the order of 20-25 nucleotides in length. Accordingly, these molecules require no further cleavage to enable their functionality in the RNA interference pathway.
(iv) short hairpin RNA (shRNA)—these molecules are also known as “small hairpin RNA” and are typically similar in length to the siRNA molecules but with the exception that they comprise inverted repeat sequences of an RNA molecule, the inverted repeats being separated by a nucleotide spacer. Subsequently to the cleavage of the hairpin (loop) region, a functional siRNA molecule is generated.
(v) micro RNA/small temporal RNA (miRNA/stRNA)—miRNA and stRNA are generally understood to represent naturally-occurring, endogenously expressed molecules. Accordingly, although the design and administration of a molecule intended to mimic the activity of a miRNA will take the form of a synthetically generated or recombinantly expressed siRNA molecule, the present invention nevertheless extends to the design and expression of oligonucleotides intended to mimic miRNA, pri-miRNA or pre-miRNA molecules by virtue of exhibiting essentially identical RNA sequences and overall structure. Such recombinantly generated molecules may be referred to as either miRNAs or siRNAs.
(vi) miRNAs which mediate spatial development (sdRNAs), the stress response (srRNAs) or cell cycle (ccRNAs).
(vii) RNA oligonucleotides designed to hybridise and prevent the functioning of endogenously expressed miRNA or stRNA or exogenously introduced siRNA. In some embodiments, it would be appreciated that these molecules are not designed to invoke the RNA interference mechanism but, rather, prevent the upregulation of this pathway by the miRNA and/or siRNA molecules which are present in the intracellular environment. In terms of their effect on the miRNA to which they hybridise, this is reflective of more classical antisense inhibition.

Reference to an “RNA oligonucleotide” should be understood as a reference to an RNA nucleic acid, molecule which is double stranded or single stranded and is capable of either inducing an RNA interference mechanism directed to silencing the expression of a target gene. In this regard, the subject oligonucleotide may be capable of directly modulating an RNA interference mechanism or it may require further processing, such as is characteristic of (i) hairpin double stranded RNA, which requires excision of the hairpin region, (ii) longer double stranded RNA molecules which require cleavage by dicer or (iii) precursor molecules such as pre-miRNA, which similarly require cleavage. The subject oligonucleotide may be double stranded (as is typical in the context of effecting RNA interference) or single stranded (as may be the case if one is seeking only to produce a RNA oligonucleotide suitable for binding to an endogenously expressed gene).

In other embodiments, the nucleic acid molecule suppresses translation initiation, splicing at a splice donor site or splice acceptor site. In other embodiments, modification of splicing alters the reading frame and initiates nonsense mediated degradation of the transcript.

It will be appreciated that a person of skill in the art can determine the most suitable nucleic acid molecule for use in accordance with the present invention and for any given situation. For example, although it is preferable that an RNA molecule exhibits 100% complementarity to its target nucleic acid sequence, the RNA molecule may exhibit some degree of mismatch to the extent that hybridisation sufficient to induce an RNA interference response in a sequence-specific manner is enabled. Accordingly, it is preferred that the RNA molecule comprises at least 70% sequence complementarity, more preferably at least 90% complementarity and even more preferably, 95%, 96%, 97%, 98% 99% or 100% sequence complementarity with the target nucleic acid sequence.

In another example pertaining to the design of a nucleic acid molecule suitable for use in accordance with the present invention, it is within the skill of the person of skill in the art to determine the particular structure and length of the molecule, for example whether it takes the form of dsRNA, hairpin dsRNA, siRNA, shRNA, miRNA, pre-miRNA, pri-miRNA or any other suitable form as herein described. For example, it is generally understood that stem-loop RNA structures, such as hairpin dsRNA and shRNA, are typically more efficient in terms of achieving gene silencing than, for example, double stranded DNA which is generated utilising two constructs separately coding the sense and antisense RNA strands. Furthermore, the nature and length of the intervening spacer region can impact on the functionality of a given stem-loop RNA molecule. In yet another example, the choice of long dsRNA, which requires cleavage by an enzyme such as Dicer, or short dsRNA (such as siRNA or shRNA) can be relevant if there is a risk that in the context of the particular cellular environment, an interferon response could be generated, this being a more significant risk where long dsRNA is used than where short dsRNA molecules are utilised. In still yet another example, whether a single stranded or double stranded nucleic acid molecule is required to be used will also depend on the functional outcome which is sought. For example, to the extent that one is targeting an endogenously expressed miRNA with an antisense molecule, it would generally be appropriate to design a single stranded RNA oligonucleotide suitable for specifically hybridising to the subject miRNA. To the extent that it is sought to induce RNA interference, a double stranded siRNA molecule may be required. In some embodiments, this may be designed as a long dsRNA molecule which undergoes further cleavage or an siRNA.

The term “gene” is used in its broadest sense and includes cDNA corresponding to the exons of a gene. Reference herein to a “gene” is also taken to include: a classical genomic gene consisting of transcriptional and/or translational regulatory sequences and/or a coding region and/or non-translated sequences (i.e. introns, 5′- and 3′-untranslated sequences); or an mRNA or cDNA molecule corresponding to the coding regions (i.e. exons), pre-mRNA and 5′- and 3′-untranslated sequences of the gene.

Reference to “expression” is a broad reference to gene expression and includes any stage in the process of producing protein or RNA from a gene or nucleic acid molecule, from pre-transcription, through transcription and translation to post-translation.

A “cell”, as used herein, includes a eukaryotic cell (e.g., animal cell, plant cell and a cell of fungi or protists) and a prokaryotic cell (e.g., a bacterium). In one embodiment, the cell is a human cell.

The term “subject”, as used herein, means either an animal or human subject. By “animal” is meant primates, livestock animals (including cows, horses, sheep, pigs and goats), companion animals (including dogs, cats, rabbits and guinea pigs), captive wild animals (including those commonly found in a zoo environment), and aquatic animals (including freshwater and saltwater animals such as fish and crustaceans. Laboratory animals such as rabbits, mice, rats, guinea pigs and hamsters are also contemplated as they may provide a convenient test system. In some embodiments, the subject is a human subject.

By “administration” of the complex or composition to a subject is meant that the agent or composition is presented such that it can be or is transferred to the subject. There is no particular limitation on the mode of administration, but this will generally be by way of oral, parenteral (including subcutaneous, intradermal, intramuscular, intravenous, intrathecal, and intraspinal), inhalation (including nebulisation), rectal and vaginal modes.

Without being bound or limited by theory, the complex of the present invention has been found to protect the nucleic acid molecule from degradation by enzymes such as RNAse and/or DNAse.

The present invention therefore also provides a method of protecting a nucleic acid molecule form enzymatic degradation, the method comprising complexing the nucleic acid molecule with a branched polymer comprising a support moiety and at least three block co-polymer chains covalently coupled to and extending from the moiety, wherein:
(i) each of the at least three block co-polymer chains comprise (a) a cationic polymer block that is covalently coupled to a hydrophilic polymer block, or (b) a cationic polymer block that is covalently coupled to a hydrophobic polymer block, said hydrophobic polymer block being covalently coupled to a hydrophilic polymer block; and
(ii) at least one of said covalent couplings associated with each of said block co-polymer chains is biodegradable.

There is also provided use of a complex for delivering a nucleic acid molecule to a cell, the complex comprising a branched polymer and the nucleic acid molecule, the branched polymer comprising a support moiety and at least three block co-polymer chains covalently coupled to and extending from the moiety, wherein:
(i) each of the at least three block co-polymer chains comprise (a) a cationic polymer block that is covalently coupled to a hydrophilic polymer block, or (b) a cationic polymer block that is covalently coupled to a hydrophobic polymer block, said hydrophobic polymer block being covalently coupled to a hydrophilic polymer block; and
(ii) at least one of said covalent couplings associated with each of said block co-polymer chains is biodegradable.

The present invention further provides use of a complex for silencing gene expression, the complex comprising a branched polymer and a nucleic acid molecule, the branched polymer comprising a support moiety and at least three block co-polymer chains covalently coupled to and extending from the moiety, wherein:
(i) each of the at least three block co-polymer chains comprise (a) a cationic polymer block that is covalently coupled to a hydrophilic polymer block, or (b) a cationic polymer block that is covalently coupled to a hydrophobic polymer block, said hydrophobic polymer block being covalently coupled to a hydrophilic polymer block; and
(ii) at least one of said covalent couplings associated with each of said block co-polymer chains is biodegradable.

In one embodiment, the nucleic acid molecule is selected from DNA and RNA. In a further embodiment, the DNA and RNA are selected from gDNA, cDNA, double or single stranded DNA oligonucleotides, sense RNAs, antisense RNAs, mRNAs, tRNAs, rRNAs, small/short interfering RNAs (siRNAs), double-stranded RNAs (dsRNA), short hairpin RNAs (shRNAs), piwi-interacting RNAs (PiRNA), micro RNA/small temporal RNA (miRNA/stRNA), small nucleolar RNAs (SnoRNAs), small nuclear (SnRNAs) ribozymes, aptamers, DNAzymes, ribonuclease-type complexes, hairpin double stranded RNA (hairpin dsRNA), miRNAs which mediate spatial development (sdRNAs), stress response RNA (srRNAs), cell cycle RNA (ccRNAs) and double or single stranded RNA oligonucleotides.

Without being bound or limited by theory, the complex of the present invention has been found to protect the nucleic acid molecule from degradation by enzymes such as RNAse and/or DNAse.

The present invention further provides use of a branched polymer in protecting a nucleic acid molecule from enzymatic degradation, the branched polymer comprising a support moiety and at least three block co-polymer chains covalently coupled to and extending from the moiety, wherein:
(i) each of the at least three block co-polymer chains comprise (a) a cationic polymer block that is covalently coupled to a hydrophilic polymer block, or (b) a cationic polymer block that is covalently coupled to a hydrophobic polymer block, said hydrophobic polymer block being covalently coupled to a hydrophilic polymer block; and
(ii) at least one of said covalent couplings associated with each of said block co-polymer chains is biodegradable.

The present invention is also directed to compositions, such as pharmaceutical compositions, comprising the nucleic acid molecule complex of the present invention. In some embodiments, the composition will comprise the nucleic acid molecule complex of the present invention and one or more pharmaceutically acceptable carriers, diluents and/or excipients.

In the compositions of the present invention, the nucleic acid molecule complex is typically formulated for administration in an effective amount. The terms “effective amount” and “therapeutically effective amount” of the nucleic acid complex as used herein typically mean a sufficient amount of the complex to provide in the course the desired therapeutic or prophylactic effect in at least a statistically significant number of subjects.

In some embodiments, an effective amount for a human subject lies in the range of about 0.1 ng/kg body weight/dose to 1 g/kg body weight/dose. In some embodiments, the range is about 1 μg to 1 g, about 1 mg to 1 g, 1 mg to 500 mg, 1 mg to 250 mg, 1 mg to 50 mg, or 1 μg to 1 mg/kg body weight/dose. Dosage regimes are adjusted to suit the exigencies of the situation and may be adjusted to produce the optimum therapeutic or prophylactic dose.

By “pharmaceutically acceptable” carrier, excipient or diluent is meant a pharmaceutical vehicle comprised of a material that is not biologically or otherwise undesirable; that is, the material may be administered to a subject along with the complex of the present invention without causing any or a substantial adverse reaction.

Aspects of the present invention include methods for treating a subject for an infectious disease, an inflammatory disease, or a cancer, the method comprising administering to the subject a complex according to the invention, or a pharmaceutical composition according to the invention, to the subject.

An important feature of the branched polymers according to the present invention is the presence of the biodegradable covalent couplings which are susceptible to degradation in biological environments. Depending on the type of biodegradable covalent coupling present, oxidative, reductive, hydrolytic or enzymatic degradation pathways present in the biological environments can promote cleavage of the coupling resulting in a reduction of the branched polymer molecular weight in conjunction with a change in the molecular environment experienced by the complexed nucleic acid. For example, a branched polymer with disulphide (S—S) linkages is susceptible to reductive cleavage in the endosomal compartment of a cell. Such degradation is believed to result in dissociation of the polymer/nucleic acid molecule complex leading to more efficient release of the nucleic acid molecule and making it more readily available to take part in, for example, gene silencing. In addition, lowering of the branched polymer's molecular weight can enhance expulsion of polymer residues from the intercellular environment after delivery of the nucleic acid molecule.

As used herein, the term “alkyl”, used either alone or in compound words denotes straight chain, branched or cyclic alkyl, preferably C₁₋₂₀ alkyl, e.g. C₁₋₁₀ or C₁₋₆. Examples of straight chain and branched alkyl include methyl, ethyl, n-propyl, isopropyl, n-butyl, sec-butyl, t-butyl, n-pentyl, 1,2-dimethylpropyl, 1,1-dimethyl-propyl, and hexyl. Examples of cyclic alkyl include mono- or polycyclic alkyl groups such as cyclopropyl, cyclobutyl, cyclopentyl, cyclohexyl, cycloheptyl, cyclooctyl, cyclononyl, cyclodecyl and the like. Where an alkyl group is referred to generally as “propyl”, butyl” etc, it will be understood that this can refer to any of straight, branched and cyclic isomers where appropriate. An alkyl group may be optionally substituted by one or more optional substituents as herein defined.

The term “alkenyl” as used herein denotes groups formed from straight chain, branched or cyclic hydrocarbon residues containing at least one carbon to carbon double bond including ethylenically mono-, di- or polyunsaturated alkyl or cycloalkyl groups as previously defined, preferably C₂₋₂₀ alkenyl (e.g. C₂₋₁₀ or C₂₋₆). Examples of alkenyl include vinyl, allyl, 1-methylvinyl, and butenyl. An alkenyl group may be optionally substituted by one or more optional substituents as herein defined.

As used herein the term “alkynyl” denotes groups formed from straight chain, branched or cyclic hydrocarbon residues containing at least one carbon-carbon triple bond including ethylenically mono-, di- or polyunsaturated alkyl or cycloalkyl groups as previously defined. Unless the number of carbon atoms is specified the term preferably refers to C₂₋₂₀ alkynyl (e.g. C₂₋₁₀ or C₂₋₆). Examples include ethynyl, 1-propynyl, 2-propynyl, and butynyl isomers, and pentynyl isomers. An alkynyl group may be optionally substituted by one or more optional substituents as herein defined.

The term “halogen” (“halo”) denotes fluorine, chlorine, bromine or iodine (fluoro, chloro, bromo or iodo).

The term “aryl” (or “carboaryl”) denotes any of single, polynuclear, conjugated and fused residues of aromatic hydrocarbon ring systems (e.g. C₆₋₂₄ or C₆₋₁₈). Examples of aryl include phenyl, biphenyl, terphenyl, quaterphenyl and naphthyl. An aryl group may or may not be optionally substituted by one or more optional substituents as herein defined. The term “arylene” is intended to denote the divalent form of aryl.

The term “carbocyclyl” includes any of non-aromatic monocyclic, polycyclic, fused or conjugated hydrocarbon residues, preferably C₃₋₂₀ (e.g. C₃₋₁₀ or C₃₋₈). The rings may be saturated, e.g. cycloalkyl, or may possess one or more double bonds (cycloalkenyl) and/or one or more triple bonds (cycloalkynyl). A carbocyclyl group may be optionally substituted by one or more optional substituents as herein defined. The term “carbocyclylene” is intended to denote the divalent form of carbocyclyl.

The term “heteroatom” or “hetero” as used herein in its broadest sense refers to any atom other than a carbon atom which may be a member of a cyclic organic group. Particular examples of heteroatoms include nitrogen, oxygen, sulfur, phosphorous, boron, silicon, selenium and tellurium, more particularly nitrogen, oxygen and sulfur.

The term “heterocyclyl” when used alone or in compound words includes any of monocyclic, polycyclic, fused or conjugated hydrocarbon residues, preferably C₃₋₂₀ (e.g. C₃₋₁₀ or C₃₋₈) wherein one or more carbon atoms are replaced by a heteroatom so as to provide a non-aromatic residue. Suitable heteroatoms include O, N, S, P and Se, particularly O, N and S. Where two or more carbon atoms are replaced, this may be by two or more of the same heteroatom or by different heteroatoms. The heterocyclyl group may be saturated or partially unsaturated, i.e. possess one or more double bonds. A heterocyclyl group may be optionally substituted by one or more optional substituents as herein defined. The term “heterocyclylene” is intended to denote the divalent form of heterocyclyl.

The term “heteroaryl” includes any of monocyclic, polycyclic, fused or conjugated hydrocarbon residues, wherein one or more carbon atoms are replaced by a heteroatom so as to provide an aromatic residue. Preferred heteroaryl have 3-20 ring atoms, e.g. 3-10. Particularly preferred heteroaryl are 5-6 and 9-10 membered bicyclic ring systems. Suitable heteroatoms include, O, N, S, P and Se, particularly O, N and S. Where two or more carbon atoms are replaced, this may be by two or more of the same heteroatom or by different heteroatoms. A heteroaryl group may be optionally substituted by one or more optional substituents as herein defined. The term “heteroarylene” is intended to denote the divalent form of heteroaryl.

The term “acyl” either alone or in compound words denotes a group containing the moiety C═O (and not being a carboxylic acid, ester or amide) Preferred acyl includes C(O)—R^{e}, wherein R^{e} is hydrogen or an alkyl, alkenyl, alkynyl, aryl, heteroaryl, carbocyclyl, or heterocyclyl residue.

The term “sulfoxide”, either alone or in a compound word, refers to a group —S(O)R^{f} wherein R^{f} is selected from hydrogen, alkyl, alkenyl, alkynyl, aryl, heteroaryl, heterocyclyl, carbocyclyl, and aralkyl. Examples of preferred R^{f} include C₁₋₂₀alkyl, phenyl and benzyl.

The term “sulfonyl”, either alone or in a compound word, refers to a group S(O)₂—R^{f}, wherein R^{f} is selected from hydrogen, alkyl, alkenyl, alkynyl, aryl, heteroaryl, heterocyclyl, carbocyclyl and aralkyl.

The term “sulfonamide”, either alone or in a compound word, refers to a group S(O)NR^{f}R^{f} wherein each R^{f} is independently selected from hydrogen, alkyl, alkenyl, alkynyl, aryl, heteroaryl, heterocyclyl, carbocyclyl, and aralkyl.

The term, “amino” is used here in its broadest sense as understood in the art and includes groups of the formula NR^{a}R^{b} wherein R^{a} and R^{b} may be any independently selected from hydrogen, alkyl, alkenyl, alkynyl, aryl, carbocyclyl, heteroaryl, heterocyclyl, arylalkyl, and acyl. R^{a} and R^{b}, together with the nitrogen to which they are attached, may also form a monocyclic, or polycyclic ring system e.g. a 3-10 membered ring, particularly, 5-6 and 9-10 membered systems.

The term “amido” is used here in its broadest sense as understood in the art and includes groups having the formula C(O)NR^{a}R^{b}, wherein R^{a} and R^{b} are as defined as above.

The term “carboxy ester” is used here in its broadest sense as understood in the art and includes groups having the formula CO₂R^{g}, wherein R^{g} may be selected from groups including alkyl, alkenyl, alkynyl, aryl, carbocyclyl, heteroaryl, heterocyclyl, aralkyl, and acyl.

As used herein, the term “aryloxy” refers to an “aryl” group attached through an oxygen bridge. Examples of aryloxy substituents include phenoxy, biphenyloxy, naphthyloxy and the like.

As used herein, the term “acyloxy” refers to an “acyl” group wherein the “acyl” group is in turn attached through an oxygen atom.

As used herein, the term “alkyloxycarbonyl” refers to an “alkyloxy” group attached through a carbonyl group. Examples of “alkyloxycarbonyl” groups include butylformate, sec-butylformate, hexylformate, octylformate, decylformate, cyclopentylformate and the like.

As used herein, the term “arylalkyl” refers to groups formed from straight or branched chain alkanes substituted with an aromatic ring. Examples of arylalkyl include phenylmethyl (benzyl), phenylethyl and phenylpropyl.

As used herein, the term “alkylaryl” refers to groups formed from aryl groups substituted with a straight chain or branched alkane. Examples of alkylaryl include methylphenyl and isopropylphenyl.

In this specification “optionally substituted” is taken to mean that a group may or may not be substituted or fused (so as to form a condensed polycyclic group) with one, two, three or more of organic and inorganic groups, including those selected from: alkyl, alkenyl, alkynyl, carbocyclyl, aryl, heterocyclyl, heteroaryl, acyl, aralkyl, alkaryl, alkheterocyclyl, alkheteroaryl, alkcarbocyclyl, halo, haloalkyl, haloalkenyl, haloalkynyl, haloaryl, halocarbocyclyl, haloheterocyclyl, haloheteroaryl, haloacyl, haloaryalkyl, hydroxy, hydroxyalkyl, hydroxyalkynyl, hydroxyalkynyl, hydroxycarbocyclyl, hydroxyaryl, hydroxyheterocyclyl, hydroxyheteroaryl, hydroxyacyl, hydroxyaralkyl, alkoxyalkyl, alkoxyalkenyl, alkoxyalkynyl, alkoxycarbocyclyl, alkoxyaryl, alkoxyheterocyclyl, alkoxyheteroaryl, alkoxyacyl, alkoxyaralkyl, alkoxy, alkenyloxy, alkynyloxy, aryloxy, carbocyclyloxy, aralkyloxy, heteroaryloxy, heterocyclyloxy, acyloxy, haloalkoxy, haloalkenyloxy, haloalkynyloxy, haloaryloxy, halocarbocyclyloxy, haloaralkyloxy, haloheteroaryloxy, haloheterocyclyloxy, haloacyloxy, nitro, nitroalkyl, nitroalkenyl, nitroalkynyl, nitroaryl, nitroheterocyclyl, nitroheteroayl, nitrocarbocyclyl, nitroacyl, nitroaralkyl, amino (NH₂), alkylamino, dialkylamino, alkenylamino, alkynylamino, arylamino, diarylamino, aralkylamino, diaralkylamino, acylamino, diacylamino, heterocyclamino, heteroarylamino, carboxy, carboxyester, amido, alkylsulphonyloxy, arylsulphenyloxy, alkylsulphenyl, arylsulphenyl, thio, alkylthio, alkenylthio, alkynylthio, arylthio, aralkylthio, carbocyclylthio, heterocyclylthio, heteroarylthio, acylthio, sulfoxide, sulfonyl, sulfonamide, aminoalkyl, aminoalkenyl, aminoalkynyl, aminocarbocyclyl, aminoaryl, aminoheterocyclyl, aminoheteroaryl, aminoacyl, aminoaralkyl, thioalkyl, thioalkenyl, thioalkynyl, thiocarbocyclyl, thioaryl, thioheterocyclyl, thioheteroaryl, thioacyl, thioaralkyl, carboxyalkyl, carboxyalkenyl, carboxyalkynyl, carboxycarbocyclyl, carboxyaryl, carboxyheterocyclyl, carboxyheteroaryl, carboxyacyl, carboxyaralkyl, carboxyesteralkyl, carboxyesteralkenyl, carboxyesteralkynyl, carboxyestercarbocyclyl, carboxyesteraryl, carboxyesterheterocyclyl, carboxyesterheteroaryl, carboxyesteracyl, carboxyesteraralkyl, amidoalkyl, amidoalkenyl, amidoalkynyl, amidocarbocyclyl, amidoaryl, amidoheterocyclyl, amidoheteroaryl, amidoacyl, amidoaralkyl, formylalkyl, formylalkenyl, formylalkynyl, formylcarbocyclyl, formylaryl, formylheterocyclyl, formylheteroaryl, formylacyl, formylaralkyl, acylalkyl, acylalkenyl, acylalkynyl, acylcarbocyclyl, acylaryl, acylheterocyclyl, acylheteroaryl, acylacyl, acylaralkyl, sulfoxidealkyl, sulfoxidealkenyl, sulfoxidealkynyl, sulfoxidecarbocyclyl, sulfoxidearyl, sulfoxideheterocyclyl, sulfoxideheteroaryl, sulfoxideacyl, sulfoxidearalkyl, sulfonylalkyl, sulfonylalkenyl, sulfonylalkynyl, sulfonylcarbocyclyl, sulfonylaryl, sulfonylheterocyclyl, sulfonylheteroaryl, sulfonylacyl, sulfonylaralkyl, sulfonamidoalkyl, sulfonamidoalkenyl, sulfonamidoalkynyl, sulfonamidocarbocyclyl, sulfonamidoaryl, sulfonamidoheterocyclyl, sulfonamidoheteroaryl, sulfonamidoacyl, sulfonamidoaralkyl, nitroalkyl, nitroalkenyl, nitroalkynyl, nitrocarbocyclyl, nitroaryl, nitroheterocyclyl, nitroheteroaryl, nitroacyl, nitroaralkyl, cyano, sulfate, phosphate, triarylmethyl, triarylamino, oxadiazole, and carbazole groups. Optional substitution may also be taken to refer to where a —CH₂— group in a chain or ring is replaced by a group selected from —O—, —S—, —NR^{a}—, —C(O)— (i.e. carbonyl), —C(O)O— (i.e. ester), and —C(O)NR^{a}— (i.e. amide), where R^{a} is as defined herein.

The invention will now be described with reference to the following non-limiting examples.

### EXAMPLES

### Materials

N,N-Dimethylaminoethyl methacrylate (DMAEMA) and oligo(ethylene glycol) methyl ether methacrylate (OEGMA₄₇₅, Mn˜475 g mol⁻¹) monomers were purchased from Aldrich and purified by stirring in the presence of inhibitor-remover for hydroquinone or hydroquinone monomethyl ether (Aldrich) for 30 min prior to use. α,α′-Azobis(isobutyronitrile) (AIBN) (TCI) was recrystallised twice from methanol prior to use. 1,1′-Azobis(cyclohexanecarbonitrile) (VAZO-88) initiator (DuPont) was used as received. N,N-Dimethylformamide (DMF) (AR grade, Merck) was degassed by sparging nitrogen for at least 15 min prior to use. Dicholormethane (DCM), n-heptane, diisopropyl ether, methyl iodide and methanol were commercial reagents and used without further purification. Pentaerythritol tetrakis(3-mercapto propionate) (Aldrich), 2-mercaptoethanol (Aldrich), 2-mercaptopyridine (Aldrich) were used as received. Hydrogen peroxide (30%) (BDH Chemicals) was used as received.

### Multi-Arms RAFT Agents

The 4-arm star RAFT agent (5) was prepared according to the procedure described below.

Four-arm star RAFT Agent (5):

(4S,4′S)-10,10-bis((R)-14-cyano-14-methyl-3,11-dioxo-16-thioxo-2,10-dioxa-6,7,15,17-tetrathianonacosyl)-7,13-dioxo-8,12-dioxa-3,4,16,17-tetrathianonadecane-1,19-diyl bis(4-cyano-4-(((dodecylthio)carbonothioyl)thio)pentanoate) (5)

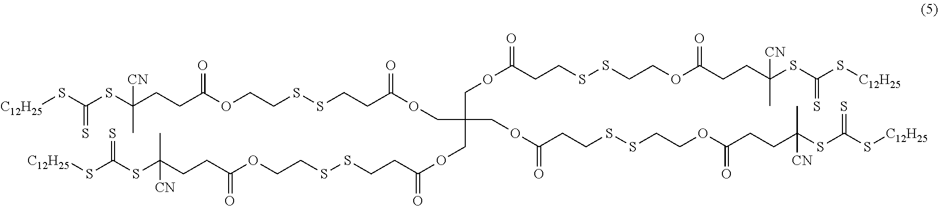

The synthetic scheme for this 4-arm star RAFT agent (5) is as following.

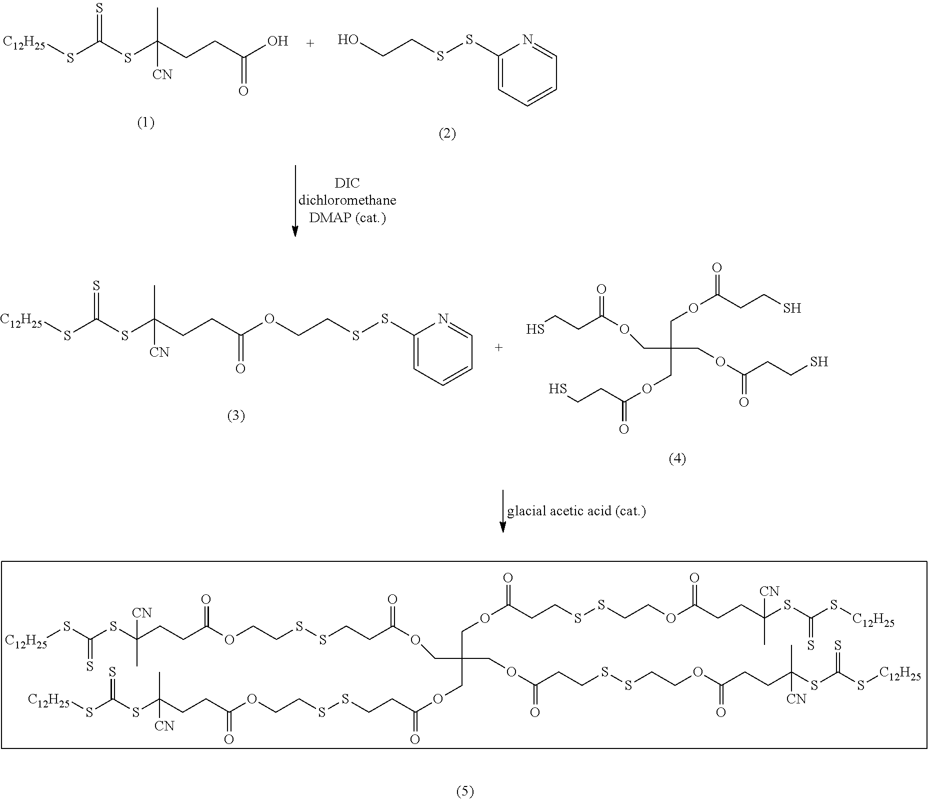

### Step 1: Synthesis of (S)-2-(pyridin-2-yldisulfanyl)ethyl 4-cyano-4-((dodecylthio)carbonothioyl)thio)pentanoate (3)

This title compound (3) was synthesized by the reaction of RAFT acid, (S)-4-cyano-4-(dodecylthiocarbonothioylthio)pentanoic acid (1) and 2-(pyridin-2-yldisulfanyl)ethanol (2) in the presence of DIC coupling agent and catalytic amount of DMAP in dichloromethane solvent.

RAFT acid (1) was prepared according to the literature procedure described in Polymer 2005, 46, 8458-8468. Compound (I) can also be purchased from Sigma-Aldrich or Strem Chemicals.

2-(Pyridin-2-yldisulfanyl)ethanol (2) was prepared according to procedure described by S. Thayumanavan et al. in Macromolecules, 2006, 39, 5595-5597.

(S)-4-cyano-4-(dodecylthiocarbonothioylthio)pentanoic acid (1) (4.60 g, 11.39 mmol), 2-(pyridine-2-yldisulfanyl)ethanol (2) (2.13 g, 11.39 mmol), DIC (diisopropylcarbodiimide, 1.58 g, 12.53 mmol) in dichloromethane (50 mL) and DMAP(N,N-dimethylaminopyridine, catalytic amount) were allowed to stir at room temperature for three hours. After removal of solvent, the crude reaction mixture was purified by column chromatography on a silica column using ethyl acetate:n-hexane 1:3 (v/v) as the eluent to give the title product (3) (5.5 g, 84% yield) as a yellow oil. ¹H NMR (CDCl₃) □ (ppm) 0.85 (t, 3H, CH₃); 1.27 (br s, 18H); 1.68 (m, 2H); 1.85 (s, 3H, CH₃); 2.35-2.60 (m, 4H, CH₂CH₂); 3.05 (t, 2H, CH₂SS); 3.35 (t, 2H, CH₂S); 4.35 (t, 2H, CH₂O); 7.10 (m, 1H, ArH); 7.65 (m, 2H, 2×ArH); 8.45 (m, 1H, ArH). ¹³C NMR (CDCl₃) □ (ppm) 14.1, 22.7, 24.9, 27.6, 28.9, 29.0, 29.3, 29.4, 29.5, 29.6 (2C), 31.9, 33.7, 37.1 (2C), 46.3, 62.7, 118.9, 119.9, 120.9, 137.0, 149.8, 159.5, 171.1, 216.0.

### Step 2: Synthesis of (5)

(S)-2-(Pyridin-2-yldisulfanyl)ethyl 4-cyano-4-(((dodecylthio)carbonothioyl)thio)pentanoate (3) (0.47 g, 8.22×10⁻⁴ mol) from step 1 above was reacted with pentaerythritol tetrakis(3-mercaptopropionate) (4) (purchased from Aldrich Chemicals) (0.10 g, 2.04×10⁻⁴ mol) in dichloromethane solvent (25 mL) with two drops of glacial acetic acid. The reaction was allowed to stir at room temperature overnight. After removal of solvent, the crude reaction mixture was purified by column chromatography on a silica column first using ethyl acetate:n-hexane 1:3 (v/v) as the eluent to remove some unreacted (3), then using ethyl acetate:n-hexane 2:3 (v/v) solvent to isolate the desired title product (5) (0.36 g, 75.5% yield) as a yellow oil. ¹H NMR (CDCl₃) □ (ppm) 0.86 (t, 12H, 4×CH₃); 1.27-1.40 (br s, 72H, 4×(CH₂)₉); 1.70 (m, 8H, 4×CH₂); 1.86 (s, 12H, 4×CH₃); 2.35 (dd, 4H, 4×CHCCN); 2.55 (dd, 4H, 4×CHCCN); 2.65 (m, 8H, 4×CH₂); 2.76 (m, 8H, 4×CH₂); 2.90 (m, 16H, 4×CH₂SSCH₂); 3.30 (t, 8H, 4×CH₂SC(═S)); 4.15 (s, 8H, 4×OCH₂C); 4.35 (t, 8H, 4×CH₂O).

### Six-Arm Star RAFT Agent (6):

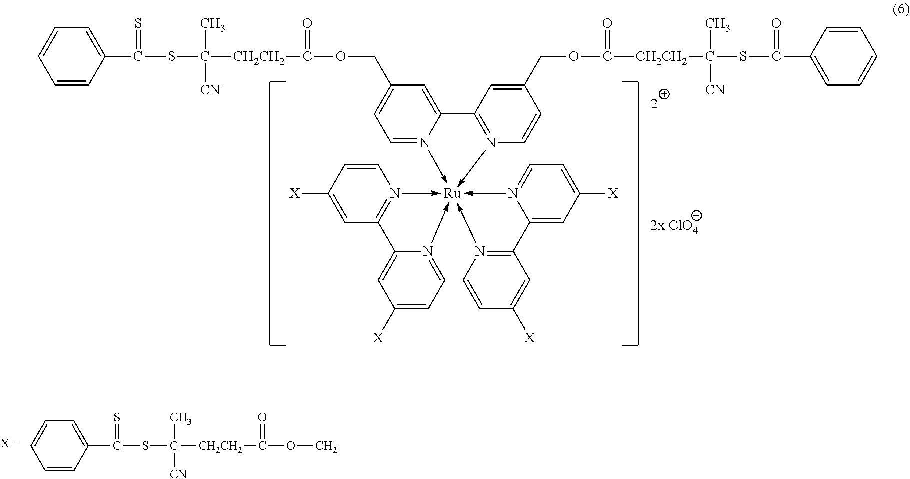

RAFT agent (6) was prepared according to literature procedure by Chen et al. published in J. Mater. Chem., 2003, 13, 2696-2700.

### Examples of Multi-Arms Star RAFT Polymers

### Example 1

### Four-Arms Star Block Copolymer ABA-B4S-16/24 (TL48A)

### ABA-B4S-16/24: PEG-DMAEMA-PEG (TL48A)

### Procedure:

PEG-DMAEMA-PEG 4-arm block copolymer

Synthesis and characterization of poly(N,N-dimethylaminoethyl methacrylate) (PDMAEMA) telechelic macroRAFT agent:

In a typical polymerization experiment, 786 mg of DMAEMA monomer (5.00×10⁻³ mol), 0.66 mg of VAZO-88 initiator (2.70×10⁻⁶ mol), 92.32 mg of 4-arm RAFT agent (5) (3.96×10⁻⁵ mol) and 620 mg of DMF (8.49×10⁻³ mol) were weighed into a Schlenk flask. The solution mixture was degassed with four freeze-evacuate-thaw cycle and polymerized at 90° C. for 5 hours.

The monomer to polymer conversion was 78% as determined by ¹H-NMR (in CDCl₃). The conversion was calculated by adding an internal standard 1,3,5-trioxane to the polymerization solution at an amount of 5 mg/1 mL. ¹H-NMR spectra before and after polymerized were compared; the integration of the —OCH₂ cyclic of the trioxane at 5.1 ppm was compared to that of the integration of the CH₂═C protons of the monomer at 5.5-6 ppm. The molecular weight of the polymer calculated based on ¹H-NMR was 17.4 kDa corresponds to a degree of polymerization of 98. The number average molecular weight (Mₙ) of the polymer as determined by gel permeation chromatography (GPC) against linear polystyrene standards was 12.8 kDa (dispersity of 1.3).

The polymer obtained was dissolved in a small amount of DCM and precipitated into heptane; the recovered polymer was then precipitated two more times using the same procedure and dried to a constant weight in a vacuum oven at 40° C.

Step 2:

Synthesis and characterization of poly(oligo(ethylene glycol) methyl ether methacrylate)-block-poly(N,N-dimethylaminoethyl methacrylate)-block-poly(oligo(ethylene glycol) methyl ether methacrylate) (P(OEGMA₄₇₅-b-DMAEMA-b-OEGMA₄₇₅): (ABA-B4S-16/24) (sample code: TL48A)

In a typical polymerization experiment, 281 mg of PDMAEMA telechelic macroRAFT agent (1.62×10⁻⁵ mol) from step 1, 712 mg of OEGMA475 (monomer) monomer (1.50×10⁻³ mol), 0.792 mg of VAZO-88 initiator (3.24×10⁻⁶ mol), and 6940 mg of DMF (9.50×10⁻² mol) were weighed into a Schlenk flask. The solution mixture was degassed with four freeze-evacuate-thaw cycle and polymerized at 90° C. for 12 hours.

The monomer to polymer conversion was 68% as determined by ¹H-NMR (in CDCl3). The conversion was calculated by adding an internal standard 1,3,5-trioxane to the polymerization solution at an amount of 5 mg/1 mL. ¹H-NMR spectra before and after polymerized were compared; the integration of the —OCH₂ cyclic of the trioxane at 5.1 ppm was compared to that of the integration of the CH₂═C protons of the monomer at 5.5-6 ppm. The molecular weight based on ¹H-NMR was 47.7 kDa. The number average molecular weight (Mₙ) of the polymer was 50.2 kDa (dispersity of 1.2) as determined by gel permeation chromatography (GPC) against linear polystyrene standards.

The polymer obtained was dissolved in a small amount of DCM then precipitated into diisopropyl ether, the polymer recovered was then precipitated two more times using the same procedure and dried to a constant weight in a vacuum oven at 40° C.

### Quarternization of the ABA Block

In a round bottom flask, ABA block copolymer was dissolved in methanol at 10% (w/v). Two mole equivalent of methyl iodide with respect to PDMAEMA portion in the block copolymer was then added, the reaction mixture stirred at room temperature for 4 hours. All volatiles were removed by rotary evaporator and then further dried in vacuum oven at 40° C.

### Dialysis

Further purification of the polymeric material was carried out by dialysis (molecular weight cut-off of 3500, Spectra Por, Spectrum Medical Industries, Inc., Houston, Tx) against de-ionized water for 3 days. After dialysis, the water was removed from the polymer solution by lyophilisation.

### Example 2

### Four-Arms Star Block Copolymer BAB-B4S-22/10 (TL68B)

### BAB-134S-22/10: DMAEMA-PEG-DMAEMA (TL68B)

### Procedure:

Synthesis and Characterization Telechelic macroRAFT of Poly(Oligo(Ethylene Glycol) Methyl Ether Methacrylate Agent:

In a typical polymerization experiment, 880 mg of OEGMA475 monomer (1.85×10⁻³ mol), 2.94 mg of VAZO-88 initiator (1.21×10⁻⁵ mmol), 137.00 mg of 4-arms RAFT agent (5) (5.88×10⁻⁵ mol) and 9818 mg of DMF (1.34×10⁻¹ mol) were weighed into a Schlenk flask. The solution mixture was degassed with four freeze-evacuate-thaw cycle and polymerized at 90° C. for 5 hours.

The monomer to polymer conversion was 46% as determined by ¹H-NMR (in CDCl₃). The conversion was calculated by adding an internal standard 1,3,5-trioxane to the polymerization solution at an amount of 5 mg/1 mL. ¹H-NMR spectra before and after polymerization were compared; the integration of the —OCH₂ cyclic of the trioxane at 5.1 ppm was compared to that of the integration of the CH₂═C protons of the monomer at 5.5-6 ppm. The molecular weight calculated based on ¹H-NMR was 20.3 kDa which corresponds to a degree of polymerization of ˜40 (average 10 OEGMA475 units per arm). The number average molecular weight (Mₙ) of the polymer was 10.9 kDa (dispersity of 1.15) as determined by gel permeation chromatography (GPC) against linear polystyrene standards.

The polymer obtained was dissolved in a small amount of DCM then precipitated into diisopropyl ether; the polymer recovered was precipitated twice using the same procedure and then dried to a constant weight in a vacuum oven at 40° C.

Step 2:

Synthesis and characterization of poly(N,N-dimethylaminoethyl methacrylates):—block-poly(oligo(ethylene glycol) methyl ether methacrylate-block-poly(N,N-dimethylaminoethyl methacrylates) [P(DMAEMA-b-OEGMA₄₇₅-b-DMAEMA)]: (BAB-B4S-22/10) or (sample code: TL68B)

In a typical polymerization experiment, 241 mg of POEGMA475 telechelic macroRAFT agent (1.62×10⁻⁵ mol) from step 1, 346 mg of DMAEMA monomer (2.20×10⁻³ mol), 1.16 mg of VAZO-88 initiator (4.75×10⁻⁶ mol), and 7202 mg of DMF (9.87×10⁻² mol) were weighed into a Schlenk flask. The solution mixture was degassed with four freeze-evacuate-thaw cycle and polymerized at 90° C. for 4.5 hours.

The monomer to polymer conversion was 56.8% as determined by ¹H-NMR (in CDCl₃). The conversion was calculated by adding an internal standard 1,3,5-trioxane to the polymerization solution at an amount of 5 mg/1 mL. ¹H-NMR spectra before and after polymerized were compared; the integration of the —OCH₂ cyclic of the trioxane at 5.1 ppm was compared to that of the integration of the CH₂═C protons of the monomer at 5.5-6 ppm. The molecular weight calculated based on NMR was 36.9 kDa. The number average molecular weight (Mₙ) of the polymer was 7.0 kDa (dispersity of 1.23) as determined by gel permeation chromatography (GPC) against linear polystyrene standards.

The polymer obtained was dissolved in a small amount of DCM then precipitated into diisopropyl ether, the precipitation obtained was then precipitated two more times then dried to a constant weight in a vacuum oven at 40° C.

### Quarternization of the ABA Block

In a round bottom flask, BAB block copolymer (BAB-B4S-22/10 or TL68B) was dissolved in methanol at 10% (w/v). Two mole equivalent of methyl iodide with respect to PDMAEMA portion in the block copolymer was then added, the reaction mixture stirred at room temperature for 4 hours. All volatiles were removed by rotary evaporator and then further dried in vacuum oven at 40° C.

### Dialysis

Further purification of the polymeric material was carried out by dialysis (molecular weight cut-off of 3500, Spectra Por, Spectrum Medical Industries, Inc., Houston, Tx) against de-ionized water for 3 days. After dialysis, the water was removed from the polymer solution by lyophilisation.

TABLE 1
Molecular weight, dispersity and composition of the
star block copolymers prepared using RAFT polymerization.

| Polymer | | | Mₙ (NMR) | Composition | |
|---|---|---|---|---|---|
| code | Mₙ (kDa) | Dispersity | (kDa) | A:B*/arm | Block |
| TL48A | 50.3 | 1.20 | 47.7 | 24:16 | ABA |
| TL48B | 8.5 | 1.2 | 9.0 | 11:10 | BAB |
| TL68B | 7.0 | 1.23 | 36.9 | 22:10 | BAB |
| TL65B1 | 13.8 | 1.4 | 52.3 | 38:10 | BAB |

*A: DMAEMA; B: OEGMA475

### Example 3

### rAB-6S (Statistical DMAEMA and OEGMA475 6-Arms Star Copolymer) (Sample code: LN2009/1735/79Q)

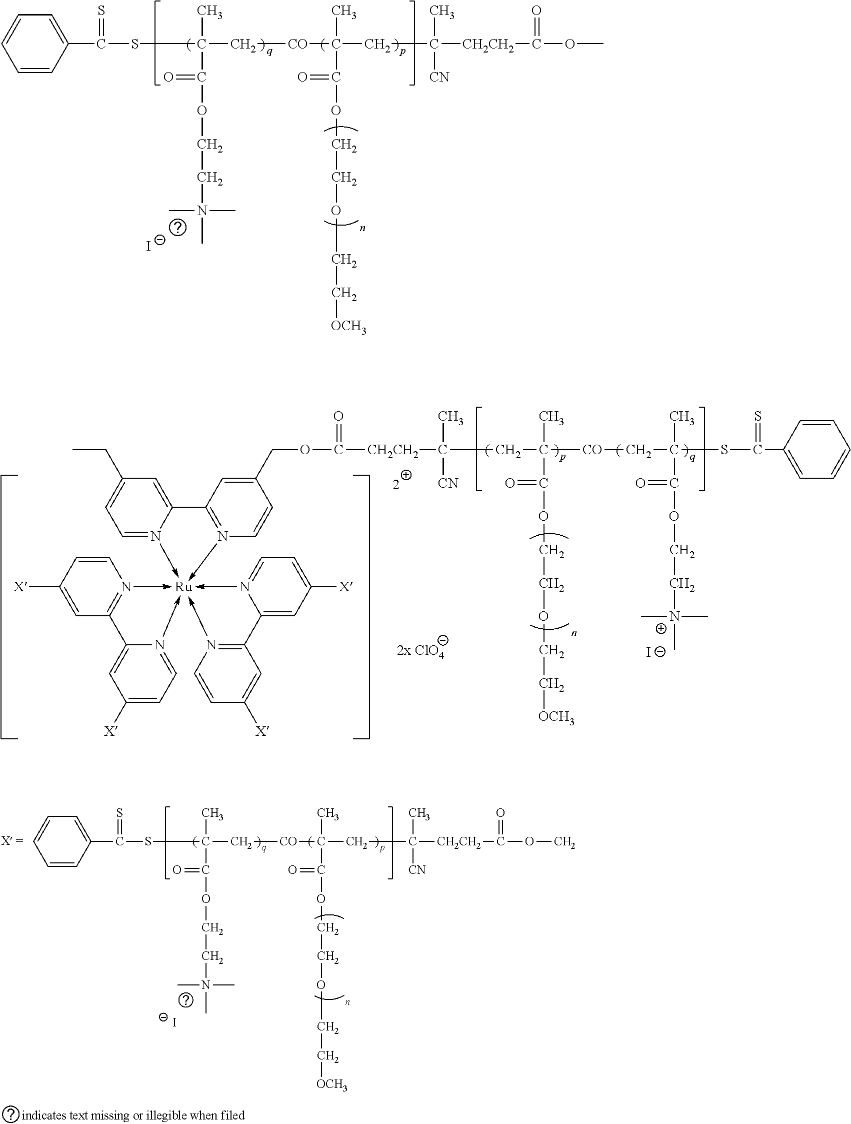

### Procedure:

RAFT Copolymerization in the Presence of Six-Arm Star RAFT Agent (6)

In a 5 mL volumetric flask, DMAEMA (1.01 g, 2.13×10⁻³ mol), OEGMA475 (0.5 g, 3.18×10⁻³ mol), AIBN (4.5 mg, 2.74×10⁻⁵ mol) and six-arm RAFT agent (6) (7.41 mg, 2.95×10⁻⁶ mol) were dissolved in DMF solvent to the 5 mL mark. This mixture was transferred to a reaction vessel and degassed by three freeze-evacuate-thaw cycles, then sealed under vacuum and heated at 60° C. for 16 hours. The polymer was purified by dialysis in MiniQ water for 48 hours. The number average molecular weight (Mₙ) of the polymer was 53.1 kDa (dispersity of 1.86) as determined by gel permeation chromatography (GPC) against linear polystyrene standards and with N,N-dimethyl acetamide as solvent.

### Quarternization of the Statistical Six-Arm Star Copolymer

In a round bottom flask, six-arm star block copolymer was dissolved in methanol at 10% (w/v). Excess methyl iodide with respect to PDMAEMA portion in the block copolymer was then added, the reaction mixture stirred at room temperature for 16 hours. All volatiles were removed by rotary evaporator and then further dried in vacuum oven at 40° C.

### Example 4

Evaluation of toxicity of the RAFT block copolymers prepared in Example 1 and 2 for different cell lines

### Materials

Cells:

Chinese Hamster Ovary cells constitutively expressing Green Fluorescent protein (CHO-GFP) (kindly received from K. Wark; CSIRO CMHT Australia) were grown in MEMα modification supplemented with 10% foetal bovine serum, 10 mM Hepes, 0.01% penicillin and 0.01% streptomycin at 37° C. with 5% CO₂ and subcultured twice weekly.

Human embryonic kidney cells constitutively expressing GFP 1EK293-GFP) were grown in RPMI₁₆₄₀ supplemented with 10% foetal bovine serum, 10 mM Hepes, 2 mM glutamine, 0.01% penicillin and 0.01% streptomycin at 37° C. with 5% CO₂ and subcultured twice weekly.

Human hepatocarcinoma cells constitutively expressing GFP (Huh7-GFP) were grown in DMEM supplemented with 10% foetal bovine serum, 10 mM Hepes, 2 mM glutamine, 0.01% penicillin and 0.01% streptomycin at 37° C. with 5% CO₂ and subcultured twice weekly

Toxicity Assay:

CHO-GFP were seeded at 3×10⁴HEK293-GFP and Huh7-GFP cells were seeded at 1×10⁴ cells per well in 96-well tissue culture plates and grown overnight at 37° C. with 5% CO₂ in 200 μl standard media.

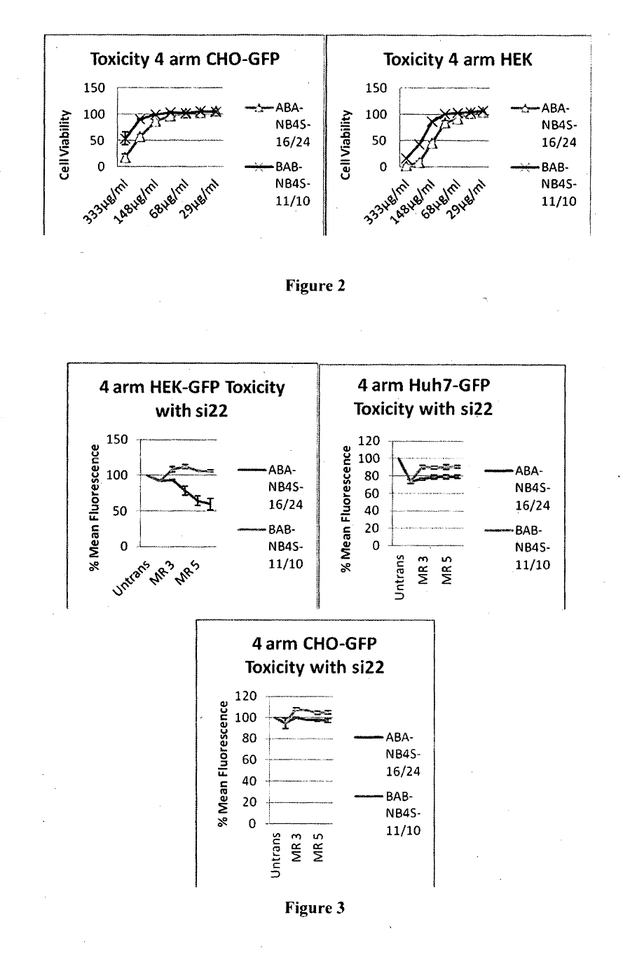

The multi-arms star RAFT block copolymer without siRNA were serially diluted in water and added to 3 wells in the 96 well culture plates for each sample then incubated for 72 h. For toxicity of samples associated with siRNA were prepared as described below in example 5. Samples were added to cells in 200 μl OPTIMEM and incubated for 5 h. The OPTIMEM was replaced with 200 μl normal media and incubated for a further 67 h. Toxicity was measured using the Alamar Blue reagent (Invitrogen USA) according to manufacturer's instructions. Briefly media was removed and replaced with 100 μl of standard media containing 10% Alamar Blue reagent, cells were then incubated for 4 h at 37° C. with 5% CO₂. The assay was read on an EL808 Absorbance microplate reader (BIOTEK, USA) at 540 nm and 620 nm. Cell viability was determined by subtracting the 620 nm measurement from the 540 nm measurement. Results are presented as a percentage of untreated cells. FIG. 2 shows the cell viability results of the multi-arm star copolymers without siRNA when tested with CHO-GFP and HEK293T cells. FIG. 3 shows the toxicity in the 3 cell lines with polymers samples associated with siRNA.

The toxicity of the polymers was investigated in two cell lines without and in three cells lines with siRNA association. CHO-GFP cells are a fast growing robust cell line, whilst HEK293T-GFP cells are more sensitive to transfection, with Huh7-GFP cells with characteristics in between the other cell lines. An acceptable toxicity level was deemed to be survival of over 70% in all cell lines. A range of polymer concentrations were analysed. BAB-B4S-22/10 (TL48B) appeared slightly more toxic than ABA-B4S-16/24 (TL48A) when analysed by serial dilution in both cell lines. BAB-B4S-22/10 (TL48B) at a MR of 4 corresponds to 30 μg/ml polymer. This concentration was not toxic with or without siRNA. ABA-B4S-16/24 (TL48A) at MR of 4 corresponds to 51 μg/ml polymer. This concentration is not toxic with polymer alone in either cell line however is toxic when associated with siRNA in HEK-GFP cells. This may be due to the internal binding of the siRNA and the different complexes that is likely to form affecting the different cells due to interaction with the membrane. Neither polymer was toxic when associated with siRNA in Huh7-GFP cells.

### Example 5

### Synthetic siRNA and DNA Oligonucleotides

The anti-GFP siRNA was obtained from QIAGEN (USA). The anti-GFP siRNA sequence is sense 5′ gcaagcugacccugaaguucau 3′ and antisense 5′ gaacuucagggucagcuugccg 3′ and is referred to as si22. Mn of the siRNA duplex is 15191 Da.

DNA oligonucleotides corresponding to anti-GFP siRNA sequence were purchased from Geneworks (Sth Australia) and are identified as di22. Oligonucleotides were annealed by combining equal molar amounts of oligonucleotides, heating to 95° C. for 10 min and gradually cooling to room temperature. These were used as negative controls as it will have no silencing effect.

Formation of Polymer/siRNA Complexes:

Molar ratios of polymer (see Table 1 for polymer Mn) to 50 nM siRNA or si22 were calculated. Complexes were formed by the addition of OPTIMEM media (Invitrogen, USA) to eppendorf tubes. The required amount of polymer resuspended to 10 mg/ml in water was added to the tubes and the mixture vortexed. 50 nM of si22 or di22 was then added to the tubes and the sample vortexed. Complexation was allowed to continue for 1 h at RT.

Agarose Gel Electrophorosis:

Samples at different molar ratios of polymer to 50 nM siRNA were electrophoresed on a 2% agarose gel in TBE at 100V for 40 min. siRNA was visualised by gel red (Jomar Bioscience) on a UV transilluminator with camera, the image was recorded by the GeneSnap program (Syngene, USA).

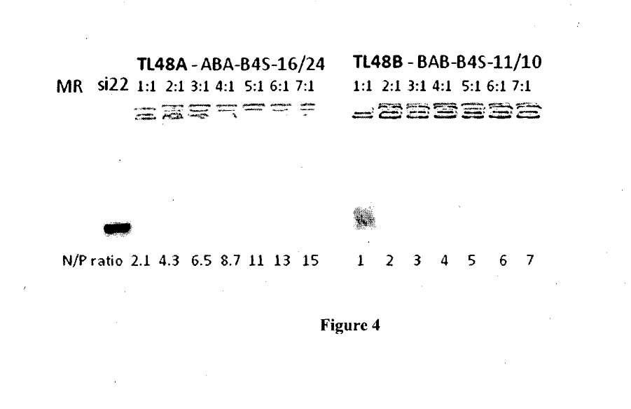

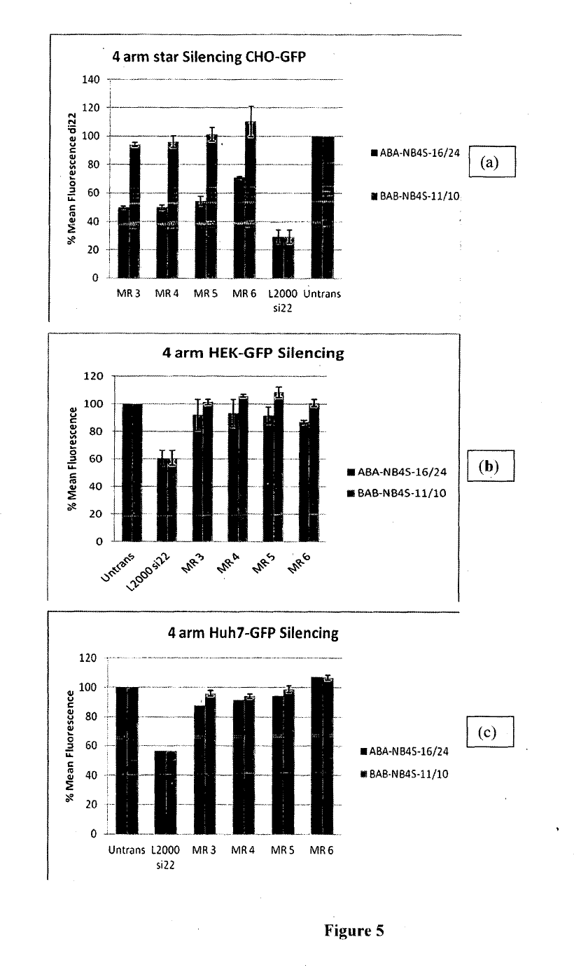

Previous work has shown that 50 nM of si22 is enough to visualise on an agarose gel and to silence 80% of the EGFP signal in CHO-GFP cells by Lipofectamine 2000 transfection (Data not shown). This amount of si22 was therefore used to determine the ability of the polymer to bind the siRNA and to silence EGFP expression in the CHO-GFP cells. Molar ratios of polymer to si22 ranging from 1:1 to 7:1 were formulated for each polymer. This was to ensure a level of polymer below the toxicity limit was used. These samples were subjected to electrophoresis and minimal differences in the ability to associate with the siRNA were observed (FIG. 4). siRNA association was determined by the shift of the siRNA from the expected 22 nt migration to being unable to enter the gel to any significant extent. Both multi-arm star polymers were able to bind siRNA at a low molar ratio of 2:1 although ABA-B4S-16/24 was also able to bind the siRNA at the molar ratio of 1. The size of the polymer siRNA complexes and zeta potential with and without siRNA was determined (see Example 6).

### Example 6

### Dynamic Light Scattering (DLS) and Zeta Potential Measurements

The hydrodynamic diameters (DH) of siRNA/block copolymer complexes were obtained via dynamic light scattering experiments that employed a Malvern-Zetasizer Nano Series DLS detector with a 22 mW He—Ne laser operating at i) 632.8 nm, an avalanche photodiode detector with high quantum efficiency, and an ALV/LSE-5003 multiple 6 digital correlator electronics system. Samples were prepared maintaining a N/P ratio of 4.0 and contained a minimum total mass per volume (i.e., block copolymer mass+siRNA mass per mL) of 0.5 mg/mL. To remove dust, samples were centrifuged at 14000 rpm for 10 min prior to characterization via DLS. All DH measurements were performed in triplicate at 25° C., and complex sizes were compared to those of the uncomplexed block copolymers and the pure siRNA.

Zeta-potential were measured in HEPES buffer using automated setting in standard disposable zeta-potential flow cell.

TABLE 2
Particle size and zeta potential of complxes formed
from RAFT polymers prepared in Examples 1 and siRNA.

| | | Zeta Potential (mV) | Particle Size (d · nm) |
|---|---|---|---|
| TL48A | 0 | 31.2 ± 0.83 | 2.9 ± 1.2 |
| | 4 | 29.07 ± 3.0 | 3.81 ± 1.8 |
| TL48B | 0 | 39.40 ± 1.6 | 8.91 ± 0.8 |
| | 4 | 34.77 ± 1.0 | 5.54 ± 2.1 |

Notes:
*DLS measurements showed a bimodal particle size distribution and values reported are for smaller size range which constituted nearly 99% of the particles.

### Example 7

### Silencing Assay

CHO-GFP cells were seeded at 3×10⁴ cells, Huh7-GFP and HEK-GFP cells were seeded at 1×10⁴ cells in 96-well tissue culture plates in triplicate and grown overnight at 37° C. with 5% CO₂. For positive and negative controls siRNAs were transfected into cells using Lipofectamine 2000 (Invitrogen, USA) as per manufacturer's instructions. Briefly, 50 picomole of the relevant siRNA (corresponding to 250 nM) were mixed with 1 μl of lipofectamine 2000 both diluted in 50 μl OPTI-MEM (Invitrogen, USA) and incubated at room temperature for 20 mins. The siNA: lipofectamine mix was added to cells in 100 μl OPTI-MEM and incubated for 4 h. Cell media was replaced and incubated for a further 67 h.

For polymer/siRNA complexes prepared according to Example 5 cell media was removed and replaced with 200 μl OPTI-MEM. The siNA: polymer complexes in a volume of 10 μl was added to 3 wells of cells per sample and incubated for 5 h. Cell media was replaced and cells incubated for a further 67 h.

Cells were washed twice with PBS, and read on a Biotek H1 synergy plate reader (Biotek, USA) set for excitation 488 nm and emission 516 am and EGFP silencing was analysed as a percentage of the polymer/di22 complexes mean EGFP fluorescence. The results are summarized in FIG. 5 and Table 3.

TABLE 3
The N/P ratio, % siRNA binding and silencing
efficiency and the polymer concentration for
various ratios of block copolymer/siRNA complexes.

| Molar Ratio | 3 | 4 | 5 | 6 |
|---|---|---|---|---|
| TL48A | a) 8.7 | a) 11 | a) 13 | a) 15 |
| | b) 100 | b) 100 | b) 100 | b) 100 |
| | c) 51 | c) 51 | c) 46 | c) 30 |
| | d) 38 | d) 51 | d) 63 | d) 76 |
| TL48B | a) 3 | a) 4 | a) 5 | a) 6 |
| | b) 100 | b)100 | b)100 | b)100 |
| | c) 6 | c) 4 | c) 0 | c) 0 |
| | d) 22 | d) 30 | d) 37 | d) 44 |

a) N/P ratio;
b) % siRNA binding;
c) % silencing efficiency in CHO-GFP cells;
d) polymer concentration μg/mL.

GFP cells ubiquitously express enhanced green fluorescent protein which when excited by a blue 488 nm laser emits a green signal at approximately 518 nm. This is readily detected by both fluorescence microscopy and flow cytometry. Silencing of the EGFP is therefore easily determined by a shift in the cell population on a flow cytometry plot and by a decrease in mean GFP fluorescence. BAB-B4S-22/10 (TL48B) was unable to silence GFP at any molar ratio in the three cell lines tested. ABA-B4S-16/24 (TL48A) was able to silence GFP to 51% at a molar ratio of 3:1 with no improvement with an increase in molar ratio.

### Cell Uptake

si22 was labelled with ToTo-3 dye as per manufacturer's instruction (Invitrogen, USA). Briefly 3 dye molecules per siRNA were bound in water for 1 h at room temperature. CHO-GFP cells were seeded at 1×10⁵ cells in 96-well tissue culture plates in triplicate and grown overnight at 37° C. with 5% CO₂. For positive controls labelled si22 were transfected into cells using Lipofectamine 2000 as described above. Polymer and labelled siRNA complexes were produced as described above and added to the cells. Cells were washed with PBS, trypsinised and washed twice with FACS wash (PBS with 1% FBS). Cells were subjected to flow cytometry and the ToTo-3 fluorescence at emission of 647 nm was analysed.

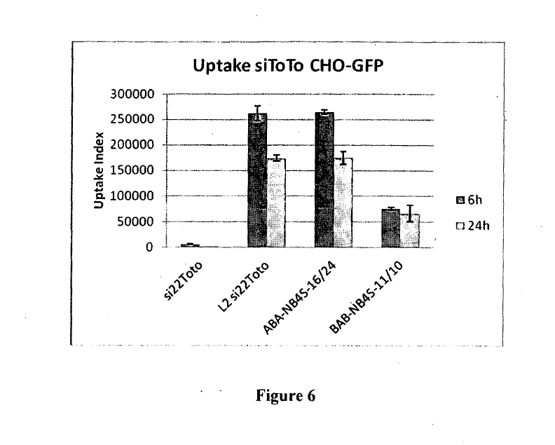

The uptake corresponds well with the silencing results for BAB-B4S-11/10 (TL48B) as the results indicate (FIG. 6) minimal uptake of the labelled siRNA and therefore polymer was observed. ABA-B4S-16/24 (TL48A) displayed good uptake of the labelled siRNA indicating delivery of the siRNA similar to lipofetamine 2000 delivery. Despite this the silencing for ABA-B4S-16/24 (TL48A) was not as efficient as the Lipofectamine 2000 indicating loss of siRNA function possibly due to degradation in the lysosome or being unable to be released from the polymer effectively.

### Example 8

### Serum Stability

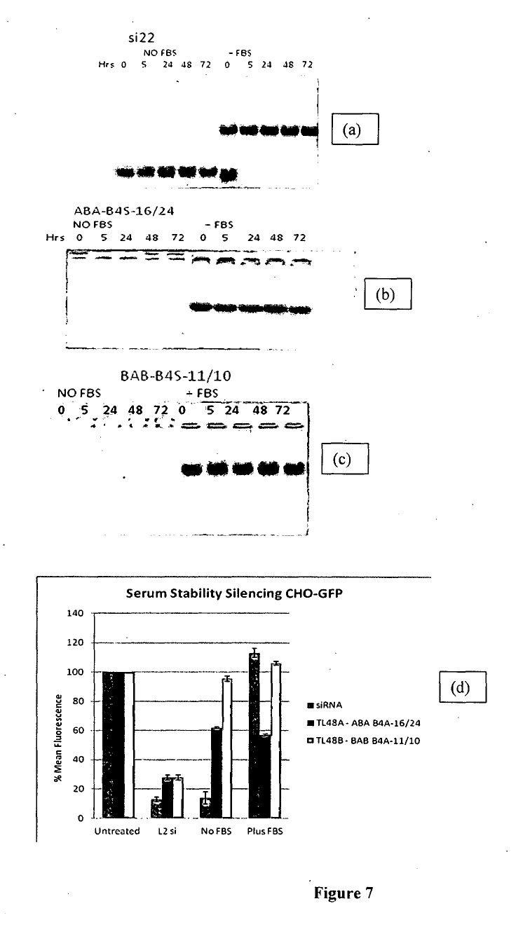

The ability of the polymer to protect the siRNA from degradation by serum proteases was performed in vitro using foetal bovine serum which is commonly used in tissue culture to provide essential growth hormones. Naked siRNA is degraded in this serum within a few hours, the results show that the siRNA contained with in the two different polymer complexes at molar ratios Of 4:1 was protected for up to 72 hours at 37° C. in 50% serum (FIG. 7). The remaining samples were then added to CHO-GFP cells to determine if the siRNA was intact and still active. Silencing was observed with ABA-B4S-16/24 (TL48A) complexes with little decrease in activity after FBS treatment (FIG. 7d). No silencing was observed with BAB-B4S-11/10 (TL48B) as expected. This is significant protection provided by the polymers. No precipitation of the complexes was observed with the serum which is also a concern as positively charged molecules are known to associate with serum proteins and precipitate out of solution (data not shown).

### Example 9

The RAFT polymer AB-B4S-16/24 was prepared using the experimental procedures described in Example 2. The toxicity, silencing assays and serum stability of this polymer was evaluated as described in Examples, 4, 7 and 8, respectively. This example illustrates the interference response of this polymer tested in chicken embryos as described below.

### IFN Response In Vivo

Commercial day 10 chicken embryos were obtained from Charles River Laboratories, Australia. Polymer complexes were injected into the allantoic cavity of a 10-day-embryonated chicken egg. The eggs were incubated at 37° C. for 6 or 24 h. PBS and si22 alone at 2 nmole were injected into eggs as controls. Allantoic membrane and liver were collected into RNA later and stored at 4°. RNA was harvested using the Trizol method.

### Reverse Transcription and Quantitative Real-Time PCR

One microgram of extracted RNA was treated with DNase (Promega, USA) according to manufacturer's instructions, quantitative real-time PCR (QRT-PCR) experiments were conducted using power Sybr green RNA to CT kit (Applied Biosystems, USA) according to manufacturer's instructions to measure cytokine expression levels. All quantification data was normalised against chicken or human GAPDH. QRT-PCR was performed on a StepOnePlus Real Time-PCR System, 96 well plate RT-PCR instrument (Applied Biosystems) under the following conditions: 1× cycle 50° C. for 30 minutes followed by 95° C. for 10 minutes, 40× cycles 95° C. for 15 seconds followed by 60° C. for 1 minute. The comparative threshold cycle (Ct) method was used to derive fold change gene expression. Primers were obtained from Geneworks (Sth Australia).

### Histopathology and Allantoic Membrane Uptake

Embryonic chicken livers were obtained from the same embryos as the membrane studied for IFN response at 24 h. Livers were fixed in 10% buffered formalin for 24 h and submitted to the pathology laboratory at the Australian Animal Health Laboratories for routine H&E staining.

Allantoic membrane was removed from embryos 24 h after injection with polymer di22-FAM complexes and fixed in 4% paraformaldehyde for 2 h. Membranes were then stained with DAPI.

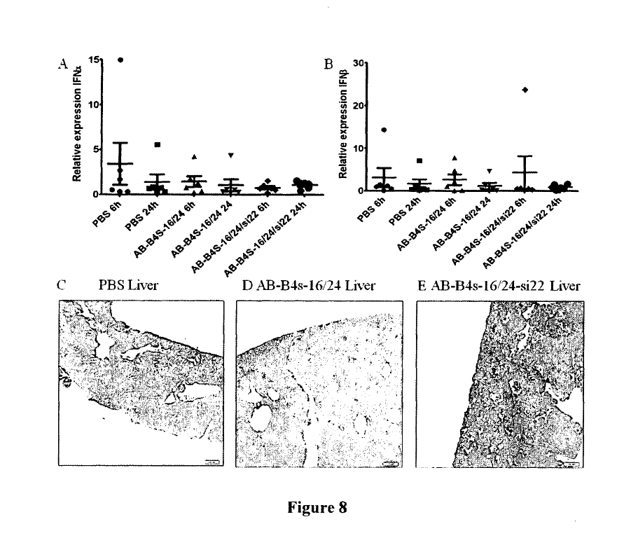

Conditions in vivo are significantly different to those in tissue culture therefore AB-B4S-16/24 was tested for toxicity, uptake and the ability to deliver an anti influenza siRNA (PB1-2257) targeting the PB1 subunit of the influenza polymerase to silence influenza replication in the chicken embryo model. Day 10 chicken embryos were injected with polymer plus or minus siRNA for 6 or 24 h, the embryo was then assayed for immune stimulation (interferon IFN α and β) by quantitative PCR and damage to the liver caused by the presence of the polymer by histopathology at 24 h. No significant increase in IFN or 13 messenger (mRNA) was observed at either the 6 or 24 hr time point in the allantoic membrane (FIGS. 8 A & B) or spleen. H & E staining by the pathology laboratory at the Australian Animal Health Laboratory also indicated no influx of immune cells or damage to the liver was observed at 24 h. This indicated the embryos were able to well tolerate the polymer over a short term period (FIGS. 8 C, D & E).

### Example 10

The RAFT polymer AB-B4S-16/24 was prepared using the experimental procedure described in Example 2. The toxicity, silencing assays and serum stability of this polymer was evaluated as described in Examples, 4, 7 and 8, respectively. This example illustrates the in-vivo influenza A-PR8 silencing by this polymer tested in chicken embryos.

### In Vivo Influenza A-PR8 Silencing

Polymer complexes were injected into the allantoic cavity of a 10-day-embryonated chicken egg. The eggs were incubated at 37° C. for 24 h. PBS was injected as a control. H1N1 Influenza PR8 virus was diluted in 100 μl PBS to 500 pfu/egg and immediately injected into the allantoic cavity of a 10-day-embryonated chicken egg. The eggs were incubated at 37° C. for 48 h and allantoic fluid was harvested to measure virus titre. Briefly, allantoic fluid was assayed for virus infectivity on MDCK cells by endpoint dilution for cytopathic effect with a 10-fold dilution series. Titres are expressed as log 10 TCID₅₀/ml.

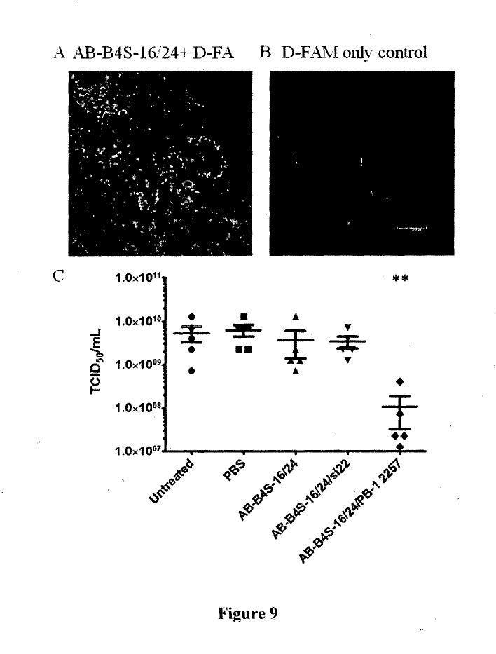

Embryos were also injected with polymer complexed with FAM labelled di22 to determine if the polymer was able to enter cells of the allantoic membrane, the main site of influenza replication, at the time of influenza injection. ABA-B4S-16/24± relevant siRNAs were injected into the allantoic fluid of day 10 embryonated chicken eggs and incubated for 24 h. 500 pfu of PR8 was injected into the allantoic fluid of each embryo and incubated at 37° C. for a further 48 h. Allantoic fluid was harvested and TCID₅₀'s performed. Results represent 5 chicken embryos per group±SEM. Statistics **p<0.01 compared to PBS. One way repeated measures ANOVA with parametric, Tukey post analysis. Strong uptake that appeared to indicate concentrated uptake in the cells surrounding the veins within the allantoic membrane and then diffusion out from these cells was observed throughout the membrane at 24 h compared to embryos injected with the FAM labelled DNA oligonucleotides alone (FIGS. 9A & B). Embryos injected with polymer alone or complexed to the irrelevant anti-GFP siRNA showed no decrease in influenza replication compared to embryos injected with PBS. Whereas embryos injected with AB-B4S-16/24 resulted in a significant average two fold decrease in virus production (FIG. 9C).

### Example 11

Materials used in Example 11 were acquired from sources described in RAFT agent synthesis Example described in this specification. Four-arm RAFT agent (5) was also synthesised as described earlier in the specification.

Synthesis and characterization of poly(N,N-dimethylaminoethyl methacrylates)-block-poly(oligo(ethylene glycol) methyl ether methacrylate-block-poly(N,N-dimethylaminoethyl methacrylates) [P(DMAEMA-b-OEGMA₄₇₅-b-DMAEMA)] without [BAB-B4S-30/16: (TL46)] and with PolyFluor [BAB-B4S-31/15 (TL47-PF)]

Step 1a: Synthesis and Characterization of Telechelic macroRAFT of Poly(Oligo(Ethylene Glycol) Methyl Ether Methacrylate Agent:

### Procedure:

In a typical polymerization experiment, 1319 mg of OEGMA475 monomer (2.78×10⁻³ mol), 3.20 mg of VAZO-88 initiator (1.31×10⁻⁵ mol), 169.60 mg of 4-arms RAFT agent (5) (7.27×10⁻⁵ mol) and 11930 mg of DMF (1.63×10⁻¹ mol) were weighed into a Schlenk flask. The solution mixture was degassed with four freeze-evacuate-thaw cycle and polymerized at 90° C. for 21 hours.

The monomer to polymer conversion was 82% as determined by ¹H-NMR (in CDCl₃). The conversion was calculated by adding an internal standard 1,3,5-trioxane to the polymerization solution at an amount of 5 mg/1 mL. ¹H-NMR spectra before and after polymerization were compared; the integration of the —OCH₂ cyclic of the trioxane at 5.1 ppm was compared to that of the integration of the CH₂═C protons of the monomer at 5.5-6 ppm. The molecular weight calculated based on ¹H-NMR was 31 kDa which corresponds to a degree of polymerization of ˜66 (average 16 OEGMA475 units per arm). The number average molecular weight (Mₙ) of the polymer was 23 kDa (dispersity of 1.25) as determined by gel permeation chromatography (GPC) against linear polystyrene standards. The polymer obtained was dissolved in a small amount of DCM then precipitated into diisopropyl ether; the polymer recovered was precipitated twice using the same procedure and then dried to a constant weight in a vacuum oven at 40° C.

Step 1b: Synthesis and Characterization Telechelic macroRAFT of Poly(Oligo(Ethylene Glycol) Methyl Ether Methacrylate Agent with PolyFluor™ 570:

In a typical polymerization experiment, 1359 mg of OEGMA475 monomer (2.86×10⁻³ mol), 3.20 mg of VAZO-88 initiator (1.31×10⁻⁵ mol), 168.7 mg of 4-arms RAFT agent (5) (7.23×10⁻⁵ mol), 19.8 mg of PolyFlour™ 570 (2.9×10⁻⁵ mol) and 11930 mg of DMF (1.63×10⁻¹ mol) were weighed into a Schlenk flask. The solution mixture was degassed with four freeze-evacuate-thaw cycle and polymerized at 90° C. for 21 hours.

The monomer to polymer conversion was 78% as determined by ¹H-NMR (in CDCl₃). The conversion was calculated by adding an internal standard 1,3,5-trioxane to the polymerization solution at an amount of 5 mg/1 mL. ¹H-NMR spectra before and after polymerization were compared; the integration of the —OCH₂ cyclic of the trioxane at 5.1 ppm was compared to that of the integration of the CH₂═C protons of the monomer at 5.5-6 ppm. The molecular weight calculated based on ¹H-NMR was 30 kDa which corresponds to a degree of polymerization of ˜62 (average 16 OEGMA475 units per arm). The number average molecular weight (Mₙ) of the polymer was 22 kDa (dispersity of 1.25) as determined by gel permeation chromatography (GPC) against linear polystyrene standards. The polymer obtained was dissolved in a small amount of DCM then precipitated into diisopropyl ether; the polymer recovered was precipitated twice using the same procedure and then dried to a constant weight in a vacuum oven at 40° C.

Step 2:

Synthesis and characterization of poly(N,N-dimethylaminoethyl methacrylates)-block-poly(oligo(ethylene glycol) methyl ether methacrylate-block-poly(N,N-dimethylaminoethyl methacrylates) [P(DMAEMA-b-OEGMA₄₇₅-b-DMAEMA)]: BAB-B4S-30/16: (TL46) and BAB-B4S-31/15: (TL47-PF)

The following procedure was used to prepare TL46 and TL47-PF)

In a typical polymerization experiment, 1503 mg of POEGMA475 telechelic macroRAFT agent (4.797×10⁻⁵ mol) from step 1a or (with PolyFlour™ 570) 1b, 1448 mg of DMAEMA monomer (9.21×10⁻³ mol), 0.586 mg of VAZO-88 initiator (2.4×10⁻⁶ mol), and 7229 mg of DMF (9.89×10⁻² mol) were weighed into a Schlenk flask. The solution mixture was degassed with four freeze-evacuate-thaw cycle and polymerized at 90° C. for 16 hours.

The monomer to polymer conversion was 63% as determined by ¹H-NMR (in CDCl₃). The conversion was calculated by adding an internal standard 1,3,5-trioxane to the polymerization solution at an amount of 5 mg/1 mL. ¹H-NMR spectra before and after polymerized were compared; the integration of the —OCH₂ cyclic of the trioxane at 5.1 ppm was compared to that of the integration of the CH₂═C protons of the monomer at 5.5-6 ppm. The molecular weight calculated based on NMR and as determined by gel permeation chromatography (GPC) against linear polystyrene standards as per Table 1. The polymer obtained was dissolved in a small amount of DCM then precipitated into diisopropyl ether, the precipitation obtained was then precipitated two more times then dried to a constant weight in a vacuum oven at 40° C.

### Quarternization Block Copolymers

In a round bottom flask, BAB block copolymer (BAB-B4S-30/16 or TL46) was dissolved in methanol at 10% (w/v). Two mole equivalent of methyl iodide with respect to PDMAEMA portion in the block copolymer was then added, the reaction mixture stirred at room temperature overnight. All volatiles were removed by rotary evaporator and then further dried in vacuum oven at 40° C.

### Dialysis

Further purification of the polymeric material was carried out by dialysis (molecular weight cut-off of 3500, Spectra Por, Spectrum Medical Industries, Inc., Houston, Tx) against de-ionized water for 3 days. After dialysis, the water was removed from the polymer solution by lyophilisation.

TABLE 4
Molecular weight, dispersity and composition of the star
block copolymers prepared using RAFT polymerization.

| | | | Mₙ | | |
|---|---|---|---|---|---|
| | Mₙ | | (NMR) | Composition | |
| Polymer code | (kDa) | Dispersity | (kDa) | A:B*/arm | Block |
| LN2012/1TL46 | 22 | 1.35 | 50.3 | 30:16 | BAB |
| LN2012/1TL47 | 21.7 | 1.31 | 48.9 | 31:15 | BAB.1% |
| | | | | | PF |

*A: DMAEMA; B: OEGMA475

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A branched polymer comprising a support moiety and at least three block co-polymer chains covalently coupled to and extending from the moiety, wherein:
(i) each of the at least three block co-polymer chains comprise (a) a cationic polymer block that is covalently coupled to a hydrophilic polymer block, or (b) a cationic polymer block that is covalently coupled to a hydrophobic polymer block, said hydrophobic polymer block being covalently coupled to a hydrophilic polymer block; and
(ii) at least one of said covalent couplings associated with each of said block copolymer chains is biodegradable.

2. The branched polymer according to claim 1, wherein each of the at least three block co-polymer chains comprises a cationic polymer block that is covalently coupled to a hydrophilic polymer block.

3. The branched polymer according to claim 1, wherein each of the at least three block co-polymer chains comprises two hydrophilic polymer blocks and a cationic polymer block, where the cationic polymer block is (i) located in between, and (ii) covalently coupled to, each of the two hydrophilic polymer blocks.

4. The branched polymer according to claim 1, wherein each of the at least three block co-polymer chains comprises a hydrophilic polymer block and two cationic polymer blocks, where the hydrophilic polymer block is (i) located in between, and (ii) covalently coupled to, each of the two cationic polymer blocks.

5. The branched polymer according to claim 1, wherein each of the at least three block co-polymer chains comprises a cationic polymer block that is covalently coupled to a hydrophobic polymer block, the hydrophobic polymer block itself being covalently coupled to a hydrophilic polymer block.

6. The branched polymer according to claim 1, wherein each of the at least three block co-polymer chains is covalently coupled to the support moiety through a covalent coupling that is biodegradable.

7. The branched polymer according to claim 1, wherein (i) the at least one of said covalent couplings associated with each of said block co-polymer chains that is biodegradable is a biodegradable linking moiety comprising one or more functional groups selected from ester, anhydride, carbonate, peroxide, peroxyester, phosphate, thioester, urea, thiourethane, ether, disulfide, carbamate (urethane) and boronate ester, and (ii) biodegradation of the one or more functional groups causes the covalent coupling to be severed.

8. The branched polymer according to claim 1 having a structure represented by formulae (A7) or (A8):
SM-(-(LM)ₓ-A-(LM)ₓ-B-(LM)ₓ-A)ᵥ (A7)
SM-(-(LM)ₓ-B-(LM)ₓ-A-(LM)ₓ-B)ᵥ (A8)
where SM represents the support moiety, LM represents a linking moiety, A represents a hydrophilic polymer block, B represents a cationic polymer block, each x is independently 0 or 1, and v is an integer greater than or equal to 3, such that (i) A, B, optionally together with LM, represent a block co-polymer arm of the branched polymer, and (ii) in each of the at least 3 block co-polymer arms at least one x=1 and the LM associated with that x=1 is a biodegradable linking moiety that covalently couples A with B.

9. A complex comprising a branched polymer and a nucleic acid molecule, the branched polymer comprising a support moiety and at least three block co-polymer chains covalently coupled to and extending from the moiety, wherein:
(i) each of the at least three block co-polymer chains comprises (a) a cationic polymer block that is covalently coupled to a hydrophilic polymer block, or (b) a cationic polymer block that is covalently coupled to a hydrophobic polymer block, said hydrophobic polymer block being covalently coupled to a hydrophilic polymer block; and
(ii) at least one of said covalent couplings associated with each of said block copolymer chains is biodegradable.

10. The complex according to claim 9, wherein the cationic polymer block comprises from about 5 to about 200 monomer residue units that each comprises positive charge.

11. The complex according to claim 9, wherein the hydrophilic polymer block comprises from about 5 to about 200 hydrophilic monomer residue units.

12. The complex according to claim 9 having a Zeta potential ranging from about 10 mV to about 40 mV.

13. The complex according to claim 9, wherein the nucleic acid molecule is selected from gDNA, cDNA, double or single stranded DNA oligonucleotides, sense RNAs, antisense RNAs, mRNAs, tRNAs, rRNAs, small/short interfering RNAs (siRNAs), double-stranded RNAs (dsRNAs), short hairpin RNAs (shRNAs), piwi-interacting RNAs (PiRNAs), micro RNA/small temporal RNA (miRNA/stRNA), small nucleolar RNAs (SnoRNAs), small nuclear RNAs (SnRNAs), ribozymes, aptamers, DNAzymes, ribonuclease-type complexes, hairpin double stranded RNA (hairpin d5RNA), miRNAs which mediate spatial development (sdRNAs), stress response RNAs (srRNAs), cell cycle RNAs (ccRNAs) and double or single stranded RNA oligonucleotides.

14. A method of delivering a nucleic acid molecule to a cell, the method comprising:
(a) providing a complex comprising a branched polymer and a nucleic acid molecule, the branched polymer comprising a support moiety and at least three block co-polymer chains covalently coupled to and extending from the moiety, wherein:
(i) each of the at least three block co-polymer chains comprises (a) a cationic polymer block that is covalently coupled to a hydrophilic polymer block, or (b) a cationic polymer block that is covalently coupled to a hydrophobic polymer block, said hydrophobic polymer block being covalently coupled to a hydrophilic polymer block; and
(ii) at least one of said covalent couplings associated with each of said block co-polymer chains is biodegradable; and
(b) delivering the complex to the cell.

15-20. (canceled)

21. The method according to claim 14, wherein the complex transfects the cell.


SEQUENCE LISTING (2 sequences)

SEQ ID NO: 1 - RNA, 22 nt, Aequorea victoria
gcaagcugacccugaaguucau

SEQ ID NO: 2 - RNA, 22 nt, Aequorea victoria
gaacuucagggucagcuugccg